(19) United States
(12) Patent Application Publication
(10) Pub. No.: US 2014/0328779 A1
(43) Pub. Date: Nov. 6, 2014

(54) COSMETIC PRODUCT CONTAINING FILM-FORMING POLYMER

(71) Applicants: SHIN-ETSU CHEMICAL CO., LTD., Tokyo (JP); SHIN-ETSU SILICONES OF AMERICA, INC., Akron, OH (US)

(72) Inventors: Mamoru HAGIWARA, Stow, OH (US); Naoki OMURA, Ridgewood, NJ (US); Masahiko MINEMURA, Annaka-shi (JP)

(21) Appl. No.: 14/334,419

(22) Filed: Jul. 17, 2014

Related U.S. Application Data
(62) Division of application No. 13/445,366, filed on Apr. 12, 2012.

(51) Int. Cl. A61K 8/81; A61Q 1/06; A61Q 5/00; A61Q 3/02

(52) U.S. Cl. CPC A61K 8/8158; A61Q 3/02; A61Q 1/06; A61Q 5/00
USPC 424/61; 424/64; 424/70.12; 424/78.35; 526/279

(57) ABSTRACT

A cosmetic product comprising a polymer having recurring units of formula (1):

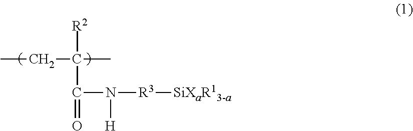

wherein R¹ is independently an alkyl group or a fluorinated alkyl group having 1 to 12 carbon atoms, R² is methyl group or hydrogen atom, R³ is a divalent saturated hydrocarbon group having 1 to 6 carbon atoms, letter a is an integer of 1 to 3, and X is a monovalent group represented by formula (i):

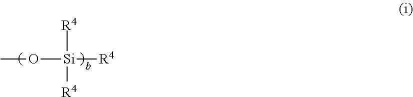

wherein R⁴ is independently an alkyl group or a fluorinated alkyl group having 1 to 12 carbon atoms and letter b is an integer of 1 to 5.

### CROSS REFERENCE

The present application is a 37 C.F.R. §1.53(b) divisional of, and claims priority to, U.S. application Ser. No. 13/445,366 filed Apr. 12, 2012. The entire contents of this application is hereby incorporated by reference.

### TECHNICAL FIELD

The present invention relates to a cosmetic product and, more particularly, to a cosmetic product containing a film-forming polymer having a polyacrylamide skeleton. The polymer has good compatibility with a variety of oils, especially silicone oils, readily spreads on skin and hair without sticky feeling, and forms a film having excellent water resistance, oil resistance and film properties. Thus, the cosmetic product of the present invention has high cosmetic function and high durability.

### BACKGROUND ART

Cosmetic products are usually incorporated with a film-forming polymer so that they keep long. For example, various silicone resins are known as the film-forming polymer (JP-A 7-196449). However, the film of the conventional film-forming polymers is low in strength, and the film is readily broken and taken off when scratched by fingers or cloths after application. Although the fragility of the film can be improved by increasing the ratio of M/Q units in the silicone resins, this solution causes the other problems that feeling becomes inferior because of sticky feeling, spreading on skin becomes worsened, and squeaking feeling occurs.

A silicone-modified acrylic ester resin is also used widely as the film-forming polymer (JP-A 2-25411). However, the silicone-modified acrylic ester resin has unsatisfied film durability and unpleasant feeling since the film imparts slimy and tacky feel.

Another known film-forming polymer is a polyacrylamide containing a siloxane (U.S. Pat. No. 4,711,943). This polymer is good in film-forming ability and finds use as gas-permeable film and contact lenses.

### SUMMARY OF THE INVENTION

Accordingly, an object of the present invention is to provide a cosmetic product comprising a film-forming polymer having good compatibility with various oils such as silicone oils, readily spreading on skin and hair without sticky feeling, and imparting good cosmetic function.

The inventors have been found that a polymer having a polyacrylamide skeleton to which a silicone is grafted as shown below can solve the problems of the conventional film-forming polymers.

Thus, the present invention provides a cosmetic product comprising recurring units of formula (1):

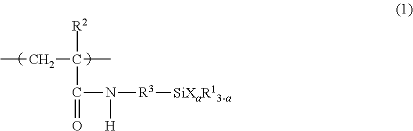

wherein R¹ is independently an alkyl group or a fluorinated alkyl group having 1 to 12 carbon atoms, R² is methyl group or hydrogen atom, R³ is a divalent saturated hydrocarbon group having 1 to 6 carbon atoms, letter a is an integer of 1 to 3, and X is a monovalent group represented by formula (i):

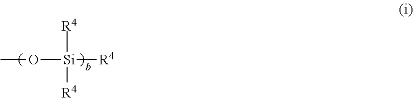

wherein R⁴ is independently an alkyl group or a fluorinated alkyl group having 1 to 12 carbon atoms and letter b is an integer of 1 to 5.

Preferably, the polymer further comprises recurring units of formula (2) and/or recurring units of formula (3):

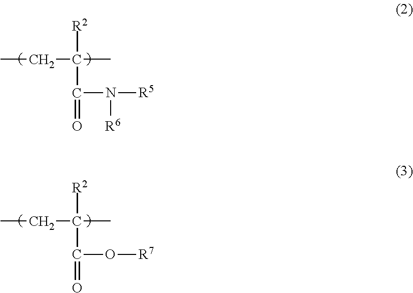

wherein R² is methyl group or hydrogen atom, R⁵ and R⁶ are independently hydrogen atom, an alkyl group, a fluorinated alkyl group, a hydroxyalkyl group, or a monovalent organic group containing a (poly)glyceryl group, or R⁵ and R⁶ may bond together to form a ring with the nitrogen atom to which they are attached, and when the ring is formed, —R⁵—R⁶— is an alkylene group which may contain an ether bond (—O—), and R⁷ is hydrogen atom, an alkyl group, a fluorinated alkyl group, a hydroxyalkyl group, or a monovalent organic group containing a (poly)oxyalkylene group, (poly)glyceryl group or siloxane bond.

The polymer preferably has a number average molecular weight of 300 to 1,000,000 by GPC using polystyrene standards.

The polymer is present preferably in an amount of 0.01 to 10% by weight, more preferably 0.1 to 7.5% by weight of the total weight of the cosmetic product.

Preferably, the cosmetic product further comprises a linear, branched or cyclic silicone oil in a weight ratio of the silicone oil and the polymer of 1:2 to 100:1.

The silicone oil is preferably volatile at 25° C., and at least one selected from the group consisting of hexamethyldisiloxane, octamethyltrisiloxane, decamethyltetrasiloxane, dodecamethylpentasiloxane, octamethylcyclotetrasiloxane, decamethylcyclopentasiloxane, tristrimethylsiloxymethylsilane, and tetrakistrimethylsiloxysilane is preferably used.

### Advantageous Effect of the Invention

The polymer of the present invention has good compatibility with various oily materials, readily spreads on skin or hair without sticky feeling, forms a thin film when applied to skin or hair, impart good feeling upon use, allows the cosmetic product to keep its effect, and has the film-forming effect which prevents the cosmetic product from staining clothes.

When applied to hair, the polymer can give good hair lustering effect, binding effect, and easy-to-comb effect.

When incorporated in a lipstick, the polymer can improve the maintenance effect on lips and give good lip lustering effect.

When incorporated in a nail enamel, the polymer can give smooth and attractive lustering effect, improve the maintenance effect, and protect nails from injuring.

### DESCRIPTION OF THE EMBODIMENTS

The following is the detailed description about the polymer and its preparation and the cosmetic products.

### (I) Polymer and its Preparation

The polymer according to the present invention which is incorporated in a cosmetic product has recurring units represented by the following formula (1):

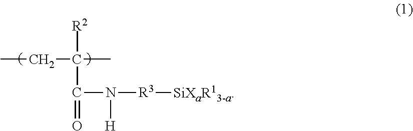

Herein R¹ is independently an alkyl group or a fluorinated alkyl group having 1 to 12 carbon atoms, preferably 1 to 6 carbon atoms. The fluorinated alkyl group is one in which at least one hydrogen atom in the alkyl group is substituted by fluorine atom. Examples of the alkyl groups and the fluorinated alkyl groups include methyl group, ethyl group, n-propyl group, butyl group, pentyl group, trifluoromethyl group, and 3,3,3-trifluoropropyl group. Among them, methyl group is preferred.

Letter a is an integer of 1 to 3, preferably 3.

The polymer may have the recurring units of formula (1) in which a=2 and the recurring units of formula (1) in which a=3 in combination.

R² is hydrogen atom or methyl group. R³ is a divalent saturated hydrocarbon group having 1 to 6 carbon atoms such as an alkylene group.

X is a monovalent linear group represented by the following formula (i):

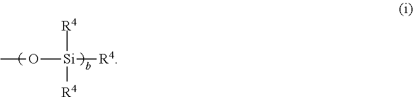

Herein, R⁴ is independently an alkyl group or a fluorinated alkyl group having 1 to 12 carbon atoms, preferably 1 to 6 carbon atoms. Examples of R⁴ include the groups exemplified in R¹. The preferable group is methyl group and trifluoromethyl group. Letter b is an integer of 1 to 5, preferably 1 to 3.

Trimethylsiloxy group shown below is preferred as the group of formula (i):

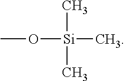

The polymer having the recurring units (1) may further comprise recurring units represented by the following formula (2) and/or recurring units represented by the following formula (3). The copolymer of the recurring units of formula (1) and the recurring units of formula (2) and/or the recurring units of formula (3) is a random copolymer. Preferred is a copolymer having the recurring units (1) and the recurring units (3).

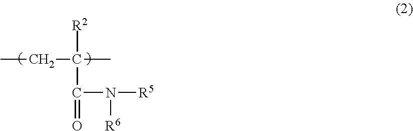

In formula (2), R² is methyl group or hydrogen atom. R⁵ and R⁶ are independently hydrogen atom, an alkyl group, a fluorinated alkyl group, a hydroxyalkyl group, or a monovalent organic group containing a (poly)glyceryl group. R⁵ and R⁶ may bond together to form a ring with the nitrogen atom to which they are attached. When the ring is formed, the group —R⁵—R⁶— is an alkylene group which may contain an ether bond (—O—).

The alkyl group, fluorinated alkyl group, hydroxyalkyl group, and alkylene group preferably have 1 to 12 carbon atoms, more preferably 1 to 6 carbon atoms. Examples of them include the groups exemplified in R¹. The monovalent organic group containing a (poly)glyceryl group includes

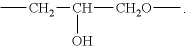

The group —R⁵—R⁶— is preferably an alkylene group having 2 to 5 carbon atoms between which an oxygen atom intervenes. Examples of the group —R⁵—R⁶— include —CH₂CH₂OCH₂CH₂—, —CH₂CH₂OCH₂CH₂CH₂—, and

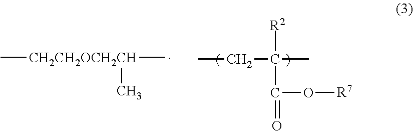

In formula (3), R² is as defined above. R⁷ is hydrogen atom, an alkyl group, a fluorinated alkyl group, a hydroxyalkyl group, or a monovalent organic group containing a (poly)oxyalkylene group, a (poly)glyceryl group or a siloxane bond. The alkyl group, fluorinated alkyl group and hydroxyalkyl group preferably have 1 to 12 carbon atoms, more preferably 1 to 6 carbon atoms. Examples of them include the groups exemplified in R¹. The monovalent organic group containing a (poly)oxyalkylene group includes —CH₂CH₂OCH₂CH₂—, —CH₂CH₂OCH₂CH₂CH₂—, and

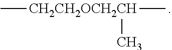

The monovalent organic group containing a (poly)glyceryl group includes

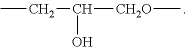

The monovalent organic group containing a siloxane bond includes the group having the following formula:

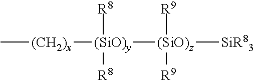

wherein letter x is an integer of 1 to 6, preferably 3, R⁸ is independently a monovalent hydrocarbon group having 1 to 8 carbon atoms such as an alkyl group or aryl group, R⁹ is R⁸ or —OSiR⁸₃ with proviso that at least one is —OSiR⁸₃, letter y is an integer of 0 to 200, preferably 10 to 100, and letter z is an integer of 0 to 50, preferably 0 to 20 with proviso that y and z are not 0 at the same time.

The recurring units (2) and/or the recurring units (3) contain preferably 5 to 50%, more preferably 10 to 20% in the total number of the recurring units. Too small ratio of the recurring units (2) and (3) would not exert the effect due to the incorporation of the recurring units (2) and (3). Too large ratio of the recurring units (2) and (3) would cause that the effect due to the recurring units (1) would not be sufficiently exerted.

The polymer used in the present invention can be produced by radical polymerizing an organic silicon compound represented by the following formula (4):

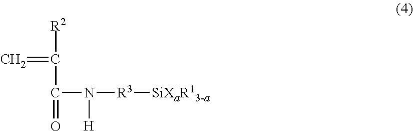

wherein R¹, R², R⁸, X and letter a are as defined above.

The organic silicon compound of formula (4) can be prepared by subjecting an amino group-containing compound of the following formula (5) to react with (meth)acrylic chloride. The detailed description of the preparation method will be given in Examples later.

H₂N—R³—SiXₐR¹₃₋ₐ (5)

wherein R¹, X, R³ and letter a are as defined above.

An example of the amino group-containing compound of formula (5) in which X is the group of formula (i) described above is a compound of formula (6) shown below.

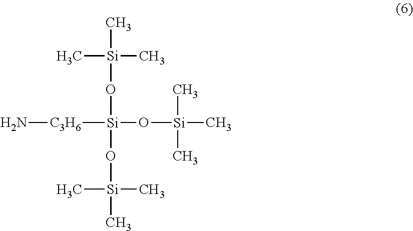

The copolymer comprising the recurring units of formula (2) and/or the recurring units of formula (3) can be obtained by radical copolymerizing the compound of formula (4) with a compound of formula (7) and/or compound of formula (8) shown below so that the compound of formula (7) and/or the compound of formula (8) is used in such an amount that the ratio of the recurring units of formula (2) and the recurring units of formula (3) is satisfied with the above-defined ratio.

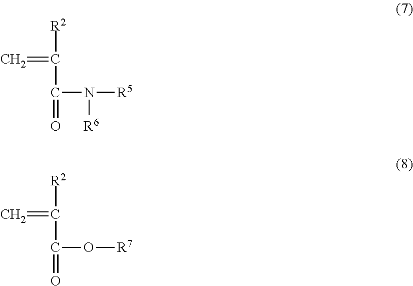

wherein R², R⁵, R⁶ and R⁷ are as defined above.

Examples of the compounds of formula (7) include the following compounds.

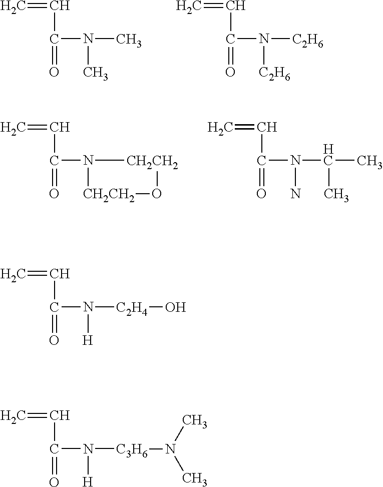

Examples of the compounds of formula (8) include alkyl (meth)acrylates such as methyl (meth)acrylate, ethyl (meth)acrylate, n-butyl (meth)acrylate and 2-ethylhexyl (meth)acrylate, hydroxyalkyl (meth)acrylate such as 2-hydroxyethyl (meth)acrylate and hydroxypropyl (meth)acrylate, perfluoroalkyl (meth)acrylate having 1 to 10 carbon atoms in which at least one hydrogen atom is substituted by fluorine atom, and silicone-modified (meth)acrylate shown below:

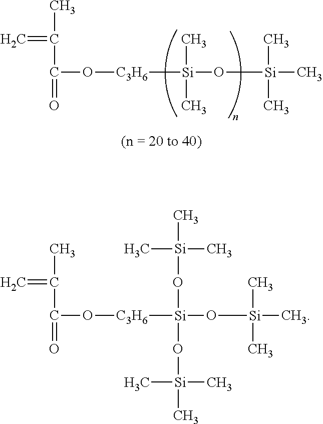

The radical polymerization is conducted in the presence of the conventional radical polymerization initiator such as benzoyl peroxide and azobisisobutyronitrile. Any method including solution polymerization method, emulsion polymerization method, suspension polymerization method, bulk polymerization method or the like can be employed. The solution polymerization method is preferred since it can easily adjust the molecular weight of the graft copolymer to an optimum range.

In the solution polymerization, a solvent is used. Examples of the solvents include aromatic hydrocarbons such as benzene, toluene and xylene, ketones such as methylethyl ketone and methylisobutyl ketone, esters such as ethyl acetate and isobutyl acetate, alcohols such as isopropanol and butanol, linear or branched dimethyl silicones having a low viscosity, and cyclic dimethyl silicones. They are used singly or in combination with one another. When the polymer is used as the form of a solution, the solution polymerization can be conducted in a suitable solvent.

The polymerization reaction can be conducted in a temperature range of 50 to 180° C., preferably 60 to 120° C. for 5 to 10 hours. The thus obtained acrylamide-silicone graft copolymer preferably has a number average molecular weight of 300 to 1,000,000, more preferably 500 to 500,000 by GPC relative to polystyrene standards using tetrahydrofuran as a solvent. If the molecular weight is too high, the viscosity of the polymer becomes higher, and when used for a cosmetic product, sticky feeling may be caused, resulting in an inferior feeling. If the molecular weight is too low, the film forming may be difficult.

The copolymer also preferably has a glass transition point between −30° C. and +60° C.

### (II) Cosmetic Product

The above-mentioned polymer (which will be referred to as ingredient-A hereinafter) is useful as a raw material for cosmetic products to be externally applied to the skin, hair, eyebrows, eyelash, and nails. Such cosmetic products include skin care cosmetics, make-up cosmetics, hair care cosmetics, antiperspirants, UV care cosmetics, and nail enamels. It is useful particularly for skin care cosmetics and make-up cosmetics which tend to cause problems with long lasting performance and secondary sticking. Ingredient-A should preferably be used in an amount of 0.01 to 10 weight % in the total amount of skin care cosmetics or UV care cosmetics, in an amount of 0.1 to 10 weight % in the total amount of cosmetic products such as foundation or in an amount of 0.1 to 7.5 weight % in the total amount of liquid cosmetic products such as milky lotion. The foregoing amount varies depending on the kind and formulation of individual cosmetic products.

The cosmetic product may be incorporated with one or more than one kind of oil as ingredient-B. This oil may be in the form of solid, semi-solid, or liquid so long as it is usable for ordinary cosmetic products.

The oil as ingredient-B includes, for example, silicone oils, silicone gums, hydrocarbon oils, higher fatty acids, higher alcohol oils, ester oils, glyceride oils, animal and vegetable oils, semi-synthetic oils, and fluorine-derived oils. They may be used alone or in combination with one another.

Examples of the silicone oils and silicone gums include, for example, straight-chain or branched-chain organopolysiloxanes ranging from low to high in viscosity such as dimethylpolysiloxane, diethylpolysiloxane, caprylyl methicone, methyl trimethicone, propyl trimethicone, phenyl trimethicone, methylphenylpolysiloxane, methylhexylpolysiloxane, methylhydrogen polysiloxane, and dimethylsiloxane-methylphenylsiloxane copolymer, cyclic organopolysiloxanes such as octamethylcyclotetrasiloxane, decamethylcyclopentasiloxane, dodecamethylcyclohexasiloxane, tetramethyltetrahydrogencyclotetrasiloxane, and tetramethyltetraphenylcyclotetrasiloxane, branched-chain organopolysiloxane such as tristrimethylsiloxymethylsilane and tetrakistrimethylsiloxysilane, amino-modified organopolysiloxane, pyrrolidone-modified organopolysiloxane, pyrrolidone carboxylic acid-modified organopolysiloxane, silicone gums such as gum-like dimethylpolysiloxane having a high degree of polymerization, gum-like amino-modified organopolysiloxane, and gum-like dimethylsiloxane-methylphenylsiloxane copolymer, solution of silicone rubber or gum in cyclic organopolysiloxane, trimethylsiloxysilicic acid, titanium oxide-containing trimethylsiloxysilicic acid, solution of trimethylsiloxysilicic acid in cyclic organopolysiloxane, higher alkoxy-modified organopolysiloxane such as stearoxysilicone, higher fatty acid-modified organopolysiloxane, alkyl-modified organopolysiloxane, long-chain alkyl-modified organopolysiloxane, amino-modified organopolysiloxane, fluorine-modified organopolysiloxane, silicone resin, and solution of silicone resin.

A portion of the oil as ingredient-B should preferably be silicone oil (volatile at room temperature 25° C.) or isododecane. Examples of the former include linear, branched, and cyclic ones, such as octamethyltrisiloxane, decamethyltetrasiloxane, octamethylcyclotetrasiloxane, decamethylcyclopentasiloxane, tristrimethylsiloxymethylsilane, and tetrakistrimethylsiloxysilane. The use of the silicone oil volatilized at room temperature (25° C.), the film of ingredient-A can be formed after the volatile silicone oil is volatilized. It is desirable that ingredient-A should be highly compatible with these silicone oils and should be used in the form of their solution prepared before addition to the cosmetic product.

The hydrocarbon oils include straight-chain ones, branched-chain ones, and volatile ones. They are exemplified by ozokerite, α-olefin oligomers, hexane, heptane, octane, cyclohexane, cyclohexene, light isoparaffin, isododecane, isohexadecane, light liquid isoparaffin, squalane, synthetic squalane, vegetable squalane, squalene, ceresin, paraffin, paraffin wax, polyethylene wax, polyethylene-polypropylene wax, (ethylene/propylene/styrene) copolymer, (butylene/propylene/styrene) copolymer, liquid paraffin, liquid isoparaffin, pristane, polyisobutylene, hydrogenated polyisobutene, microcrystalline wax, and vaseline.

Examples of the higher fatty acid include lauric acid, myristic acid, palmitic acid, stearic acid, behenic acid, undecylenic acid, oleic acid, linoleic acid, linolenic acid, arachidonic acid, eicosapentaenoic acid (EPA), docosahexaenoic acid (DHA), isostearic acid, and 12-hydroxystearic acid.

Examples of the higher alcohol oil include lauryl alcohol, myristyl alcohol, palmityl alcohol, stearyl alcohol, behenyl alcohol, hexadecyl alcohol, oleyl alcohol, isostearyl alcohol, hexyldodecanol, octyldodecanol, cetostearyl alcohol, 2-decyltetradecinol, cholesterol, phytosterol, POE cholesterol ether, monostearyl glycerine ether (batyl alcohol), and monooleyl glyceryl ether (selachyl alcohol).

Examples of the ester oils include diisobutyl adipate, 2-hexyldecyl adipate, di-2-heptylundecyl adipate, N-alkylglycol monoisostearate, isocetyl isostearate, trimethylolpropane triisostearate, ethyleneglycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, neopentylglycol dioctanoate, neopentylglycol dicaprylate, triethyl citrate, 2-ethylhexyl succinate, amyl acetate, ethyl acetate, butyl acetate, isocetyl stearate, butyl stearate, diisopropyl sebacate, di-2-ethylhexyl sebacate, cetyl lactate, myristyl lactate, isononyl isononanoate, isotridecyl isononanoate, isopropyl palmitate, 2-ethylhexyl palmitate, 2-hexyldecyl palmitate, 2-heptylundecyl palmitate, cholesteryl 12-hydroxystearylate, dipentaerythritol fatty acid ester, isopropyl myristate, octyldodecyl myristate, 2-hexyldecyl myristate, myristyl myristate, hexyldecyl dimethyloctanoate, ethyl laurate, hexyl laurate, N-lauroyl-L-glutamic acid-2-octyldodecyl ester, lauroylsarcosine isopropyl ester, and diisostearyl malate.

Examples of the glyceride oils include acetoglyceryl, glyceryl triisooctanoate, glyceryl triisostearate, glyceryl triisopalmitate, glyceryl monostearate, glyceryl di-2-heptylundecanoate, glyceryl trimyristate, and diglyceryl myristate and isostearate.

Examples of the animal and vegetable oils and semisynthetic oils include avocado oil, linseed oil, almond oil, purified insect wax, perilla oil, olive oil, cacao butter, kapok wax, kaya nut oil, carnauba wax, liver oil, candelilla wax, purified candelilla wax, beef tallow, neat's foot oil, neat's bone oil, hardened beef tallow, apricot kernel oil, whale oil, hardened oil, wheat germ oil, sesame oil, rice germ oil, rice barn oil, sugarcane wax, sasanqua oil, sunflower oil, shea butter, china wool oil, cinnamon oil, jojoba wax, squalane, squalene, shellac wax, turtle oil, soybean oil, tea seed oil, camellia oil, evening primrose oil, corn oil, lard, rapeseed oil, Japanese tung oil, rice bran wax, germ oil, horse tallow, persic oil, palm oil, palm kernel oil, castor oil, hardened castor oil, castor oil fatty acid methyl ester, sunflower oil, grape seed oil, bayberry wax, jojoba oil, macademianut oil, bees wax, mink oil, mink wax, meadowfoam oil, cottonseed oil, cotton wax, Japanese wax, Japanese wax kernel oil, montan wax, coconut oil, hardened coconut oil, coconut fatty acid triglyceride, mutton tallow, peanut oil, lanolin, liquid lanolin, reduced lanolin, lanolin alcohol, lanolin wax, hard lanolin, lanolin acetate, lanolin acetate alcohol, lanolin fatty acid isopropyl, POE lanolin alcohol ether, POE lanolin alcohol acetate, lanolin fatty acid polyethylene glycol, POE hydrogenated lanolin alcohol ether, and egg yolk oil. (POE stands for polyoxyethylene.)

Examples of the fluorine-containing oils include perfluoropolyoxyalkylene, perfluorodecalin, and perfluorooctane.

The amount of ingredient-B should be 1 to 98 weight % in the total amount of the cosmetic product. It should be properly adjusted according to the formulation of the cosmetic product. The above-mentioned silicone oil which is volatile at 25° C. and of straight-chain, branched-chain, or cyclic structure should be added in such an amount that the ratio (by mass) of the silicone oil to the polymer as ingredient-A is from 1:2 to 100:1, preferably from 1:1 to 70:1.

The cosmetic product according to the present invention may be incorporated with one or more than one kind of compound (as ingredient-C) which contains an alcoholic hydroxyl group in its molecular structure. Examples of such a compound include lower alcohol such as ethanol, isopropanol, and butanol, sugar alcohol such as sorbitol and maltose, sterol such as cholesterol, sitosterol, phytosterol, and lanosterol, and polyhydric alcohol such as butylene glycol, propylene glycol, dibutylene glycol, and pentylene glycol. Common examples of ingredient-C are water-soluble monohydric alcohols and water-soluble polyhydric alcohols. The amount of ingredient-C should be 0.1 to 98 weight % in the total amount of the cosmetic product.

The cosmetic product according to the present invention may be incorporated with one or more than one kind of water-soluble polymeric compound or water-swelling polymeric compound as ingredient-D that suits its intended use. Examples of the water-soluble polymeric compound are listed as following. Polymeric compounds of vegetable origin, such as acacia, tragacanth, galactan, carob gum, guar gum, karaya gum, carrageenan, pectin, agar-agar, quince seed, starch (rice, corn, potato, wheat, etc.), algecolloid, trant gum, and locust bean gum. Polymeric compounds of microbe origin, such as xanthan gum, dextran, succinoglucan, and pullulan. Polymeric compounds of animal origin, such as collagen, casein, albumin, and gelatin. Polymeric compounds derived from starch, such as carboxymethyl starch and methylhydroxypropyl starch. Polymeric compounds derived from cellulose, such as methyl cellulose, ethyl cellulose, methylhydroxypropyl cellulose, carboxymethyl cellulose, hydroxymethyl cellulose, hydroxypropyl cellulose, nitrocellulose, cellulose sodium sulfate, carboxymethyl cellulose sodium, crystalline cellulose, cellulose acetate, and cellulose powder. Polymeric compounds derived from alginic acid, such as sodium alginate and propylene glycol alginage ester. Polymeric compounds having vinyl groups, such as polyvinyl methyl ether and carboxyvinyl polymer. Polymeric compounds of polyoxyethylene. Polymeric compounds of polyoxyethylene-polyoxypropylene copolymer. Acrylic polymeric compounds, such as sodium polyacrylate, polyethyl acrylate, polyacrylamide, and acryloyldimethyl taulin salt copolymer. Other synthetic water-soluble polymeric compounds, such as polyethylene imine and cationic polymer. Inorganic polymeric compounds, such as bentonite, aluminum-magnesium silicate, montmorillonite, beidellite, nontronite, saponite, hectorite, and silicic anhydride. In addition, the foregoing water-soluble polymeric compounds include such film-forming agents as polyvinyl alcohol and polyvinyl pyrrolidone. They should be preferably added in an amount of 0 to 25 weight % in the total amount of the cosmetic product.

The cosmetic product according to the present invention may be incorporated with water as ingredient-E. The amount of water, which varies depending on formulation, should be 1 to 99 weight % in the total amount of the cosmetic product.

The cosmetic product according to the present invention may be incorporated with a powder as ingredient-F. So long as being suitable for ordinary cosmetic products, this powder is not specifically restricted in shape (spherical, acicular, placoid, dendroid, fibrous, or amorphous), particle size (fume, microsphere, or pigment), and particle structure (porous, nonporous, hollow, or hollow porous). Examples of such powder include inorganic powder, organic powder, powder of metal salt of surface active agent, colored pigment, pearlescent pigment, metal powder pigment, tar dye, and natural color.

Examples of the inorganic powder include titanium oxide, zirconium oxide, zinc oxide, cerium oxide, magnesium oxide, barium sulfate, calcium sulfate, magnesium sulfate, calcium carbonate, magnesium carbonate, talk, mica, kaolin, cerisite, white mica, synthetic mica, bronze mica, lepidolite, black mica, lithia mica, silicic acid, silicic acid anhydride, aluminum silicate, magnesium silicate, aluminum magnesium silicate, calcium silicate, barium silicate, strontium silicate, metal tungstate, hydroxyapatite, vermiculite, higilite, bentonite, montmorillonite, hectolite, zeolite, ceramics powder, dibasic calcium phosphate, alumina, aluminum hydroxide, boron nitride, silicon nitride, silica, and silylated silica. Of these examples, zinc oxide and titanium oxide are preferable. In addition, calcium carbonate and talc, which are extender pigments, may also be used preferentially. The powders listed above are made to easily disperse into the cosmetic product containing ingredient-A.

Examples of the organic powders include polyamide powder, polyacrylic acid-acrylate ester powder, polyester powder, polyethylene powder, polypropylene powder, polystyrene powder, polyurethane powder, benzoguanamine powder, polymethylbenzoguanamine powder, tetrafluoroethylene powder, polymethyl methacrylate powder, cellulose powder, silk powder, powder or fibrous powder of nylon such as 12-nylon and 6-nylon, crosslinked silicone fine powder prepared by crosslinking dimethylpolysiloxane, spherical fine powder of crosslinked polymethylsilsesquioxane, spherical fine powder of crosslinked organopolysiloxane rubber (having its surface coated with polymethylsilsesquioxane), hydrophobic silica, fine laminated powder or crystalline fibrous powder of resins such as styrene-acrylic acid copolymer, divinylbenzene-styrene copolymer, vinyl resin, urea resin, phenolic resin, fluoroplastic resin, silicone resin, acrylic resin, melamine resin, epoxy resin, and polycarbonate resin, starch powder, fatty acid starch derivative powder, and lauroyl lysine.

Preferable among these examples are fine powder of crosslinked spherical dimethylpolysiloxane (which partly has the crosslinked structure of dimethylpolysiloxane), fine powder of crosslinked spherical polymethylsilsesquioxane, fine powder of crosslinked spherical dimethylpolysiloxane rubber (having its surface coated with polymethylsilsesquioxane), fine powder of crosslinked spherical diphenylpolysiloxane rubber (having its surface coated with polymethylsilsesquioxane), and hydrophobic silica. They are commercially available from Shin-Etsu Chemical Co., Ltd. under the trade names of KMP-598, KMP-400, KMP-590, KSP-100, KSP-101, KSP-102, KSP-105, KSP-300, KSP-411, and KSP-441.

Examples of the powder of metal salt of surface active agent include metal soaps, such as zinc stearate, aluminum stearate, calcium stearate, magnesium stearate, zinc myristate, magnesium myristate, zinc cetylphosphate, calcium cetylphosphate, zinc sodium cetylphosphate, zinc palmitate, aluminum palmitate, and zinc laurate.

The coloring agents include pigments and dyes. Examples of the colored pigments include inorganic red pigments such as iron oxide, iron hydroxide, and iron titanate, inorganic brown pigments such as γ-iron oxide, inorganic yellow pigments such as yellow iron oxide and Chinese yellow, inorganic black pigments such as black iron oxide and carbon black, inorganic violet pigments such as silica-coated iron pigment, manganese violet, and cobalt violet, inorganic green pigment such as chromium hydroxide, chromium oxide, cobalt oxide, and cobalt titanate, inorganic blue pigment such as prussian blue and ultramarine blue, laked tar dye, laked natural dye, and synthetic resin powder formed by combination of the foregoing powders.

Examples of the pearlescent pigments include mica coated with titanium oxide, bismuth oxychloride, bismuth oxychloride coated with titanium oxide, talk coated with titanium oxide, silica-coated mica titanium, fish scale guanine, and colored mica with titanium oxide coating.

Examples of the metal powders include aluminum powder, copper powder, and stainless steel powder.

Examples of the tar dyes include red 3, red 104, red 106, red 201, red 202, red 204, red 205, red 220, red 226, red 227, red 228, red 230, red 401, red 505, yellow 4, yellow 5, yellow 202, yellow 203, yellow 204, yellow 401, blue 1, blue 2, blue 201, blue 404, green 3, green 201, green 204, green 205, orange 201, orange 203, orange 204, orange 206, and orange 207. Examples of the natural dyes include carminic acid, laccaic acid, carthamin, brazilin, and crocin.

Additional examples of the powders include fine powder of titanium oxide, fine powder of iron-containing titanium oxide, fine powder of zinc oxide, fine powder of cerium oxide, and a combination thereof, which absorb or scatter ultraviolet rays. These powders may have surface treatment with aluminum oxide, aluminum hydroxide, silica, silica gel, zinc silicate, or the like, for the purpose of blocking the surface activity of the powder.

The foregoing powders may be combined with other powders or surface-treated with an adequate material to such an extent that the present invention is not adversely affected. Examples of the surface-treatment materials include ordinary oils, silicone oils, fluorine compounds, surface active agents, and metal soaps. Additional examples are listed as following. Alkyl group having a hydrogen atom directly attaching to a silicon atom or a hydrolyzable silyl group. Linear and/or branched organopolysiloxane having a hydrogen atom directly attaching to a silicon atom or a hydrolyzable silyl group. Linear and/or branched organopolysiloxane (modified with long-chain alkyl groups) having a hydrogen atom directly attaching to a silicon atom or a hydrolyzable silyl group. Linear and/or branched organopolysiloxane (modified with polyoxyalkylene) having a hydrogen atom directly attaching to a silicon atom or a hydrolyzable silyl group. Acrylsilicone copolymer having a hydrogen atom directly attaching to a silicon atom or a hydrolyzable silyl group. They may be used alone or in combination with one another according to need. These powders may be used after surface treatment with any of AES-3083, KF-99P, KF-9901, KF-9908, KF-9909, and KP-574, all available from Shin-Etsu ChEmical Co., Ltd.

The powder may be preferably added in an amount of 0.5 to 20 weight %, more preferably 1 to 10 weight % in the total amount of the cosmetic product.

The cosmetic product according to the present invention may be incorporated with a surface active agent as ingredient-G. The surface active agent falls under the category of anionic, cationic, nonionic, and amphoteric. They are not specifically restricted so long as they are intended for cosmetic use.

Examples of the anionic surface active agents include fatty acid soaps such as sodium stearate, potassium stearate, triethanolamine stearate, sodium palmitate, potassium palmitate, triethanolamine palmitate, sodium laurate, potassium laurate, and triethanolamine laurate; alkylether carboxylic acid and salt thereof; salt of condensate of amino acid and fatty acid; alkane sulfonate, alkene sulfonate, sulfonate of fatty acid ester, sulfonate of fatty acid amide, sulfonate of formalin condensate, alkyl sulfate ester salt, secondary higher alcohol sulfate ester salt, alkyl and allyl ether sulfate ester salt, sulfate ester salt of fatty acid ester, sulfate ester salt of fatty acid alkylolamide, sulfate ester salt such as sulfated castor oil, alkyl phosphate, ether phosphate, alkylallyl ether phosphate, amide phosphate, N-acyl lactate, N-acylsarcosine salt, and N-acylamino acid surfactant. Examples of the cationic surface active agents include amine salts such as alkylamine salt, and polyamine and aminoalcohol fatty acid derivatives, alkyl quaternary ammonium salt, aromatic quaternary ammonium salt, pyridinium salt, and imidazolium salt.

Examples of the nonionic surface active agents include sorbitan fatty acid ester, glycerin fatty acid ester, polyglycerin fatty acid ester, propylene glycol fatty acid ester, polyethylene glycol fatty acid ester, sucrose fatty acid ester, methylglucoside fatty acid ester, alkylpolyglucoside, polyoxyethylene alkyl ether, polyoxypropylene alkyl ether, polyoxyethylene alkyl phenyl ether, polyoxyethylene fatty acid ester, polyoxyethylene sorbitan fatty acid ester, polyoxyethylene sorbitol fatty acid ester, polyoxyethylene glycerin fatty acid ester, polyoxyethylene propylene glycol fatty acid ester, polyoxyethylene castor oil, polyoxyethylene hardened castor oil, polyoxyethylene phytostanol ether, polyoxyethylene phytosterol ether, polyoxyethylene cholestanol ether, polyoxyethylene cholesteryl ester, linear or branched polyoxyalkylene-modified organopolysiloxane, linear or branched polyoxyalkylene-alkyl comodified organopolysiloxane, linear or branched polyglycerin-modified organopolysiloxane, linear or branched polyglycerin-alkyl comodified organopolysiloxane, alkanolamide, sugar ether, and sugar amide. Examples of the amphoteric surface active agents include betaine, phosphatidylcholine, salt of aminocarboxylic acid, imidazoline derivatives, and those of amideamine type.

Preferable among the foregoing surface active agents are linear or branched organopolysiloxane having polyoxyethylene chains in the molecule, linear or branched organopolysiloxane having polyglycerin chains in the molecule, and their alkyl-modified organopolysiloxane. They are commercially available from Shin-Etsu Chemical Co., Ltd. under the trade names of KF-6011, KF-6011P, KF-6043, KF-6012, KF-6013, KF-6015, KF-6016, KF-6017, KF-6028, KF-6028P, KF-6038, KF-6100, KF-6104, and KF-6105. They should preferably have an HLB value of 1 to 10. The amount of ingredient-G should be 0.1 to 20 weight %, preferably 0.2 to 10 weight %, in the total amount of the cosmetic product.

The cosmetic product according to the present invention may be incorporated with one or more than one kind of crosslinked organopolysiloxane as ingredient-H if necessary for its intended use. The crosslinked organopolysiloxane should preferably be one which swells upon absorption of liquid oil more than its own weight. The liquid oil may be the one mentioned above as ingredient-B. It includes, for example, low-viscosity silicone oils having a viscosity of 0.65 to 100.0 mm²/s (at 25° C.), liquid paraffin, squalane, hydrocarbon oil such as isododecane, glyceride oil such as trioctanoin, ester oil such as isotridecyl isononanoate, N-acrylglutamate ester, and lauroyl sarcosinate ester, and natural animal and vegetable oils such as macadamia nut oil.

It is desirable that the crosslinked organopolysiloxane be crosslinked by a crosslinking agent which has more than one vinyl reaction site in the molecule and forms the crosslinked structure upon reaction between hydrogen atoms each joining directly to a silicon atom. Examples of the crosslinking agent include organopolysiloxane having more than one vinyl group in the molecule, polyoxyalkylene having more than one allyl group in the molecule, polyglycerin having more than one allyl group in the molecule, and α,ω-alkenyldiene.

In addition, the crosslinked organopolysiloxane may contain a moiety selected from the group composed of polyoxyalkylene moiety, polyglycerin moiety, alkyl moiety, alkenyl moiety, aryl moiety, and fluoroalkyl moiety. The crosslinked organopolysiloxane is not specifically restricted, and it is commercially available from Shin-Etsu Chemical Co., Ltd. under the trade names of KSG-15, KSG-16, KSG-18, KSG-1610, USG-102, USG-103, USG-105, USG-106, USG-107A, USG-109, USG-110, KSG-210, KSG-240, KSG-710 (in the form of paste with silicone oil), USG-106, KSG-41, KSG-42, KSG-43, KSG-44, KSG-310, KSG-320, KSG-330, KSG-340, KSG-810, KSG-820, KSG-830, KSG-840 (in the form of paste with hydrocarbon oil or triglyceride oil).

The amount of ingredient-H should be 0.01 to 40 weight %, preferably 0.1 to 30 weight %, in the total amount of the cosmetic product.

The cosmetic product according to the present invention may be incorporated with one or more than one kind of silicone resin (I). The silicone resin (I) is preferably an acryl/silicone graft or block copolymer. It is also preferably acrylsilicone resins which contain in the molecule a moiety selected from the group composed of pyrrolidone moiety, long-chain alkyl moiety, polyoxyalkylene moiety, fluoroalkyl moiety, and anionic moiety such as carboxylic acid. It is commercially available from Shin-Etsu Chemical Co., Ltd. under the trade names of KP-541, KP-543, KP-545, KP-545L, KP-549, KP-550, KP-571, and KP-575 (dissolved in silicone oil or hydrocarbon oil).

The cosmetic product according to the present invention may also be incorporated with one or more than one kind of silicone network compound or resin (II) according to the intended use. The silicone resin (II) may preferably be a silicone network compound represented by MQ, MDQ, MT, MTQ, MDT, or MDTQ, where M, D, T, and Q are respectively R₃SiO_{0.5} unit, R₂SiO unit, RSiO_{1.5} unit, and SiO₂ unit. Such units are generally used in the silicone field. The silicone network resin is known as MQ resin, MT resin, or MDT resin. It may also have moieties indicated by MDQ, MTQ, or MDTQ. It is commercially available in the form of solution in decamethylcyclopentasiloxane, dimethycone or isododecane. It is commercially available from Shin-Etsu Chemical Co., Ltd. under the trade names of KF-7312J, KF-7312K, KF-7312T, X-21-5035, KF-9021, X-21-5595, and X-21-5616 (dissolved in silicone oil, hydrocarbon oil or alcohol).

The silicone resin, such as acrylsilicone resin and silicone network compound, should be used in an amount of 0.1 to 30 weight %, preferably 1 to 20 weight %, in the total amount of the cosmetic product.

The cosmetic product according to the present invention may be incorporated with silicone wax as ingredient-J for its intended use. This silicone wax should preferably be a polylactone-modified polysiloxane in which the polylactone is a ring-opening polymerizate of lactone compound of ring structure with five or more members. It should preferably be an acryl-modified polysiloxane containing in the molecule a moiety selected from the group composed of pyrrolidone moiety, long-chain alkyl moiety, polyoxyalkylene moiety, fluoroalkyl moiety, and anion moiety such as carboxylic acid. It is commercially available from Shin-Etsu Chemical Co., Ltd. under the trade names of KP-561P and KP-562P as wax containing long-chain alkyl group.

Moreover, the above-mentioned silicone wax should preferably be silicone-modified olefin wax which is obtained by addition reaction from olefin wax and organohydrogenpolysiloxane having more than one SiH linkage in one molecule. The olefin wax should preferably be one which is obtained by copolymerization from ethylene and at least one species of diene or one which is obtained by copolymerization from ethylene, at least one species of olefin selected from C3-12 α-olefins, and at least one species of diene. A preferred diene is vinylnorbornene.

The silicone wax, no matter what, should be used in an amount of 0.1 to 20 weight %, preferably 1 to 10 weight %, in the total amount of the cosmetic.

The cosmetic product according to the present invention may be incorporated with any ingredient listed as following, which is used for ordinary cosmetic products, in an amount not harmful to the effect of the present invention. Oil-soluble gelling agent, organo-modified clay mineral, resin, antiperspirant, UV absorber, UV absorbing-scattering agent, moisturizer, antibacterial agent, perfume, salt, antioxidant, pH adjuster, chelating agent, refrigerant, antiinflammatory agent, skin care agent (whitening agent, cell activating agent, rough skin improving agent, blood flow stimulant, skin astringent, antiseborrheic agent, etc.), vitamin, amino acid, nucleic acid, hormone, inclusion compound, and hair fixing agent.

Examples of the oil-soluble gelling agent include the following. Metal soaps such as aluminum stearate, magnesium stearate, and zinc myristate; amino acid derivatives such as N-lauroyl-L-glutamic acid-α,γ-di-n-butylamine; dextrin fatty acid ester such as dextrin palmitic acid ester, dextrin stearic acid ester, and dextrin 2-ethylhexanoic acid palmitic acid ester; sucrose fatty acid ester such as sucrose palmitic acid ester and sucrose stearic acid ester; fructo-oligosaccharide fatty acid ester such as fructo-oligosaccharide stearic acid ester and fructo-oligosaccharide 2-ethylhexanoic acid ester; benzylidene derivative of sorbitol such as monobenzylidene sorbitol and dibenzylidene sorbitol; and organo-modified clay mineral such as dimethylbenzyldodecyl ammonium montmorillonite clay and dimethyldioctadecyl ammonium montmorillonite clay.

Examples of the antiperspirant include aluminum chlorohydrate, aluminum chloride, aluminum sesquichlorohydrate, zirconyl hydroxychloride, aluminum zirconium hydroxychloride, and aluminum zirconium glycine complex.

Examples of the UV absorber include those based on benzoic acid such as para-aminobenzoic acid, those based on anthranilic acid such as methyl anthranilate, those based on salicylic acid such as methyl salicylate, octyl salicylate, and trimethylcyclohexyl salicylate, those based on cinnamic acid such as octyl paramethoxycinnamate, those based on benzophenone such as 2,4-dihydroxybenzophenone, those based on urocanic acid such as ethyl urocanate, those based on dibenzoylmethane such as 4-t-butyl-4′-methoxybenzoylmethane, and those based on phenylbenzimidazole sulfonic acid. Examples of the UV absorbing-scattering agent include fine powder of titanium oxide, fine powder of iron-containing titanium oxide, fine powder of zinc oxide, fine powder of cerium oxide, and their combination, which absorb and scatter UV rays. These fine powders may be used in the form of dispersion in oil.

Examples of the moisturizers include glycerin, sorbitol, propylene glycol, dipropylene glycol, 1,3-butylene glycol, pentylene glycol, glucose, xylitol, maltitol, polyethylene glycol, hyaluronic acid, chondroitin sulfate, pyrrolidone carboxylate, polyoxyethylene methyl glucoside, polyoxypropylene methyl glucoside, egg yolk lecithin, soybean lecithin, phosphatidylcholine, phosphatidylethanolamine, phosphatidylserine, phosphatidylglycerol, phosphatidylinositol, and sphingophospholipid.

Examples of the antibacterial agents include paraoxybenzoate alkyl ester, benzoic acid, sodium benzoate, sorbic acid, potassium sorbate, phenoxyethanol, salicylic acid, phenol, parachorometacresol, hexachlorophene, benzalkonium chloride, chlorohexidine chloride, trichlorocarbanilide, and photosensitive substance.

Examples of the salts include inorganic salts, organic salts, amine salts, and amino acid salts. Examples of the inorganic salts include sodium, potassium, magnesium, calcium, aluminum, zirconium, and zinc salts of inorganic acids such as hydrochloric acid, sulfuric acid, carbonic acid, and nitric acid. Examples of the organic salts include salts of organic acids such as acetic acid, dehydroacetic acid, citric acid, malic acid, succinic acid, ascorbic acid, and stearic acid. Examples of the amine salts and amino acid salts include salts of amines such as triethanolamine and salts of amino acids such as glutamic acid. Additional examples include salts of hyaluronic acid and chondroitin sulfuric acid, aluminum zirconium glycine complex, and acid-alkali neutralized salts which are commonly used for cosmetic formulations.

Examples of the antioxidants include tocopherol, p-t-butylphenol, butylhydroxyanisole, dibutylhydroxytoluene, and phytic acid. Examples of the pH adjuster include lactic acid, citric acid, glycolic acid, succinic acid, tartaric acid, dl-malic acid, potassium carbonate, sodium hydrogen carbonate, and ammonium hydrogen carbonate. Examples of the chelating agents include alanine, sodium edetate, sodium polyphosphate, sodium metaphosphate, and phosphoric acid. Examples of the refrigerants include L-menthol and camphor. Examples of the antiinflammatory agents include allantoin, glycyrrhizic acid and salt thereof, glycyrrhetic acid, stearyl glycyrrhetinate, tranexamic acid, and azulene.

Examples of the skin care agents include whitening agents such as placenta extract, albutin, glutathione, and saxifraga sarmentosa extract, cell activating agent such as royal jelly, photosensitive substance, cholesterol derivatives, and calf blood extract, skin roughness-improving agent, blood flow stimulant such as nonylic vanillamide, nicotinic benzyl ester, nicotinic β-butoxyethyl ester, capsaicin, zingerone, cantharis tincture, ichthammol, caffeine, tannic acid, α-borneol, tocopherol nicotinate, inositol hexanicotinate, cyclandelate, cinnarizine, tolazoline, acetylcholine, verapamil, cepharanthine, and γ-oryzanol, skin astringent such as zinc oxide and tannic acid, and antiseborrheic agent such as sulfur and thiantrol.

Examples of the vitamins include vitamin A analogs such as vitamin A oil, retinol, retinol acetate, and retinol palmitate, vitamin B₂ analogs such as riboflavin, riboflavin butyrate, and flavinadenine nucleotide, vitamin B₆ analogs such as pyridoxine hydrochloride, pyridoxine dioctanoate, and pyridoxine tripalmitate, vitamin B analogs such as vitamin B₁₂ and derivatives thereof, and vitamin B₁₅ and derivatives thereof, vitamin C analogs such as L-ascorbic acid, ester of L-ascorbic acid dipalmitic acid, sodium salt of L-ascorbic acid-2-sulfuric acid, and diester and dipotassium salt of L-ascorbic acid phosphoric acid, vitamin D analogs such as ergocalciferol and cholecalciferol, vitamin E analogs such as α-tocopherol, β-tocopherol, γ-tocopherol, dl-α-tocopherol acetate, dl-α-tocopherol nicotinate, and dl-α-tocopherol succinate, vitamin H, vitamin P, nicotinic acid analogs such as nicotinic acid, benzyl nicotinate, and nicotinic amide, pantothenoic acid analogs such as calcium pantothenate, D-pantothenyl alcohol, pantothenyl ethyl ether, and acetylpantothenyl ethyl ether, and biotin.

Examples of the amino acids include glycine, valine, leucine, isoleucine, serine, threonine, phenylalanine, arginine, lysine, aspartic acid, glutamic acid, cystine, cysteine, methionine, and tryptophane. Examples of the nucleic acids include deoxyribonucleic acid. Examples of the hormones include estradiol and ethenylestradiol.

The polymeric compound to fix the cosmetic product to nails and hair includes, for example, amphoteric, anionic, cationic, and nonionic ones as listed as following. Polyvinylpyrrolidone polymers such as polyvinylpyrrolidone and vinylpyrrolidone/vinyl acetate copolymer, acidic vinyl ether polymers such as methyl vinyl ether/maleic anhydride alkyl half ester copolymer, acidic polyvinyl acetate polymers such as vinyl acetate/crotonic acid copolymer, acidic acrylic polymers such as (meth)acrylic acid/alkyl (meth)acrylate copolymer, (meth)acrylic acid/alkyl (meth)acrylate/alkylacrylamide copolymer, and amphoteric acrylic polymers such as N-methacryloylethyl-N,N-dimethylammonium•α-N-methylcarboxybetaine/alkyl (meth)acrylate copolymer and hydroxypropyl (meth)acrylate/butylaminoethyl methacrylate/acrylic acid octylamide copolymer. Additional examples include natural polymers such as cellulose and derivatives thereof, keratin, collagen, and derivatives thereof and synthetic resins such as alkyd resin, epoxy resin, phenolic resin, unsaturated polyester, acrylic copolymer, urethane resin, various rubbers such as NBR, SBR, SIS, SBS, and EPDM, and chlorinated polyethylene.

The cosmetic product according to the present invention, which is composed of the foregoing ingredients, comes in the form of powder, oil, oil-in-water emulsion, water-in-oil emulsion, nonaqueous emulsion, or multiemulsion (W/O/W and O/W/O). Examples of the form include toilet water, milky lotion, cream, cleansing, pack, massage oil, facial lotion, facial oil, cleansing agent, deodorant, hand cream, lip cream, skin care product (to hide wrinkles), make-up base, concealer, white make-up powder, powder foundation, liquid foundation, cream foundation, oil foundation, blush-on product, eye shadow, mascara, eye liner, eyebrow, lipstick, manicure products such as nail enamel, nail undercoat, and nail overcoat, hair cosmetics such as shampoo, rinse, treatment, and setting lotion, antiperspirant, and ultraviolet protective cosmetic preparations such as sunscreen oil, sunscreen milky emulsion, and sunscreen cream.

The foregoing cosmetic products may be produced in the form of liquid, milky emulsion, cream, solid, paste, gel, powder, press, layer, mousse, aerosol, spray, stick, and others.

Moreover, the foregoing cosmetic product may be applied to the skin, hair, eye brow, eye lash, and nail by means of fingers or applicators such as brush, chip, puff, and spatula.

### EXAMPLES

The invention will be described in more detail with reference to the following Examples, which are not intended to restrict the scope thereof. In the Examples, “%” means “weight %” unless otherwise mentioned.

### Production (1) of Monomer

### Synthesis of tris(trimethylsiloxy)silylpropyl acrylamide

In a reactor was charged 750 g of tristrimethylsiloxypropylamine, 2400 g of hexane, and 257 g of triethylamine. While agitating, 211 g of acrylic chloride was dropped under nitrogen atmosphere at 20 to 30° C., and then the mixture was reacted for 3 hours. After acrylic chloride in the reaction solution was inactivated, the reaction mixture was washed with 2000 g of water three times. The organic layer was taken out and hexane was distilled off under reduced pressure, thereby obtaining 770 g of the aimed tris(trimethylsiloxy)silylpropyl acrylamide. The product was found to be a compound represented by the structural formula below as the result of ¹H-NMR analysis (with LAMBDA LA-300W made by JEOL Co., Ltd.) and IR analysis (with FT-IR Spectrometer Spectrum One made by Perkin Elmer Co., Ltd.). This compound is designated as monomer-A.

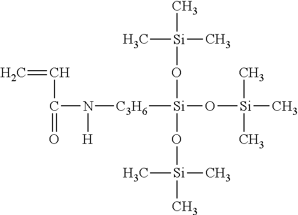

### Production (1) of Polymer

### Synthesis of tris(trimethylsiloxy)silylpropyl acrylamide polymer

In a 100 ml, four-necked flask was charged 50 g of monomer-A, which was dissolved in 2 g of hexane. Then, the atmosphere of the flask was replaced with Ar or N₂. The monomer-A solution was heated while agitating so that it is evenly diffused, and kept at 80° C. To the solution was rapidly added benzoyl peroxide as a polymerization initiator to conduct the polymerization reaction for 5 hours. The reaction solution was poured into a large amount of methanol to separate out the aimed product, which was then dried under reduced pressure. The thus obtained solid is designated as polymer-A.

The polymer-A was found to have a number average molecular weight (Mn) of 35,000 and a molecular weight distribution (Mw/Mn) of 2.34 by gel permeation chromatography (measurement apparatus: HLC-8220 manufactured by Tosoh Corporation) using polystyrene standard.

### Production (2) of Polymer

### Synthesis of tris(trimethylsiloxy)silylpropyl acrylamide-b-N-isobutyl acrylamide copolymer

The same procedure as in Production (1) of polymer was repeated except that a mixture of 45 g of monomer-A and 5 g of N-isobutyl acrylamide was used instead of 50 g of monomer-A. The thus obtained product was designated as polymer-B. The polymer-B was found to have Mn of 34,000 and Mw/Mn of 2.21 by GPC versus polystyrene standard.

### Production of polymer-C

### Synthesis of tris(trimethylsiloxy)silylpropyl acrylamide-b-N,N-diethyl acrylamide copolymer

The same procedure as in Production (1) of polymer was repeated except that a mixture of 45 g of monomer-A and 5 g of N,N-diethyl acrylamide was used instead of 50 g of monomer-A. The thus obtained product was designated as polymer-C. The polymer-C was found to have Mn of 38,000 and Mw/Mn of 2.4 by GPC versus polystyrene standard.

Solution of polymer-A in decamethylcyclopentasiloxane (D5)

In a separable flask with its atmosphere replaced by nitrogen were placed 40 g of polymer-A and 93.5 g of D5. After dissolution at 80° C. by a glass stirrer, there was obtained a 30% homogeneous solution. The viscosity of the solution was about 200 mPa·s.

### Solution of Polymer-A in Methyltrimethicone (M3T)

The same procedure as above was repeated except that M3T was used as a solvent instead of D5.

### Solution of Polymer-A in Isododecane

The same procedure as above was repeated except that isododecane was used as a solvent instead of D5.

### Solution of Polymer-B in M3T

In a separable flask with its atmosphere replaced by nitrogen were placed 40 g of polymer-B and 93.5 g of M3T. After dissolution at 80° C. by a glass stirrer, there was obtained a 30% homogeneous solution.

### Solution of Polymer-C in D5

In a separable flask with its atmosphere replaced by nitrogen were placed 40 g of polymer-C and 93.5 g of D5. After dissolution at 80° C. by a glass stirrer, there was obtained a 30% homogeneous solution.

Examples of cosmetic products in which the above polymers or their solutions were used are shown below.

### Example 1 and Comparative Examples 1 and 2

Each example demonstrates the cream foundation of emulsion type having the composition shown in Table 1.

TABLE 1

| | | | Comparative | |
|---|---|---|---|---|
| | Composition | Emample | Example | |
| No. | (weight %) | 1 | 1 | 2 |
| 1 | Crosslinked polyether- | 5.0 | 5.0 | 5.0 |
| | modified silicone (*1) | | | |
| 2 | Crosslinked | 6.0 | 6.0 | 6.0 |
| | dimethylpolysiloxane (*2) | | | |
| 3 | Polyether-modified silicone (*3) | 1.0 | 1.0 | 1.0 |
| 4 | Dimethylpolysiloxane | 3.0 | 3.0 | 3.0 |
| | (6 mm²/sec at 25° C.) | | | |
| 5 | Decamethylcyclopentasiloxane | 5.0 | 6.2 | 5.0 |
| 6 | Triethylhexanoin | 5.0 | 5.0 | 5.0 |
| 7 | Neopentylglycol dioctanoate | 2.0 | 2.0 | 2.0 |
| 8 | Polymethylsilsesquioxane | 1.5 | 1.5 | 1.5 |
| | powder (*4) | | | |
| 9 | 1,3-BG | 5.0 | 5.0 | 5.0 |
| 10 | Sodium chloride | 0.5 | 0.5 | 0.5 |
| 11 | Water | 50.0 | 50.0 | 50.0 |
| 12 | Silicone-treated titanium oxide (*5) | 8.65 | 8.65 | 8.65 |
| 13 | Silicone-treated red iron oxide (*5) | 0.45 | 0.45 | 0.45 |
| 14 | Silicone-treated yellow | 0.75 | 0.75 | 0.75 |
| | iron oxide (*5) | | | |
| 15 | Silicone-treated black | 0.15 | 0.15 | 0.15 |
| | iron oxide (*5) | | | |
| 16 | Solution of polymer-A in D5 | 3.0 | — | — |
| 17 | Silicone resin (*6) | — | 1.8 | — |
| 18 | Silicone-modified acrylic | — | — | 3.0 |
| | polymer (*7) | | | |
| 19 | Antioxidant | q.s. | q.s. | q.s. |
| 20 | Antiseptic | q.s. | q.s. | q.s. |
| 21 | Perfume | q.s | q.s. | q.s. |

| | | | |
|---|---|---|---|
| Total | 100.0 | 100.0 | 100.0 |

*1: KSG-210 from Shin-Etsu Chemical Co., Ltd.
*2: KSG-15 from Shin-Etsu Chemical Co., Ltd.
*3: KF-6017 from Shin-Etsu Chemical Co., Ltd.
*4: KMP-561P from Shin-Etsu Chemical Co., Ltd.
*5: treated with KF-9909 from Shin-Etsu Chemical Co., Ltd.
*6: KF-7312J (50 wt % solution of silicone resin in D5) from Shin-Etsu Chemical Co., Ltd.
*7: KP-545 (30 wt % solution of silicone-modified acrylic polymer in D5) from Shin-Etsu Chemical Co., Ltd.

### [Preparation of Cosmetic Product]

Ingredients (1) to (8), (19), and (20) were uniformly mixed by stirring. To the resulting mixture was slowly added for emulsification a homogenous solution of ingredients (9) and (10) dissolved in ingredient (11). The emulsified product was mixed with ingredients (12) to (18) and (21) which had been rolled previously. The resulting mixture was filled into prescribed containers. Thus there was obtained the cream foundation of emulsion type as desired. This cosmetic product was evaluated in the following way.

### [Evaluation of Usability]

The cream foundation of emulsion type which was obtained as mentioned above was rated for usability by 50 female expert panelists according to the following criteria.

Items of usability rated: Ease of spreading, sticky feel, unevenness, transparency, and lasting quality.

The results are shown below in terms of average.

### [Effect of Preventing Secondary Sticking]

Each sample was applied in a similar manner to the forehead of the panelist, and, 10 minutes after application, a piece of tissue paper was pressed against the forehead to see if secondary sticking occurs. The sample was rated for the effect of preventing secondary sticking according to the following criteria. The results are shown below in terms of average.

TABLE 2
Rating criteria

| | | | | | | Secondary |
|---|---|---|---|---|---|---|
| Point | Spreadability | Sticking | Unevenness | Transparency | Lasting | sticking |
| 5 | Good | None | None | Transparent | Good | None |
| 4 | Slightly | Almost none | Almost none | Slightly | Fair | Almost none |
| | good | | | transparent | | |
| 3 | Moderate | Moderate | Moderate | Moderate | Moderate | Moderate |
| 2 | Slightly | Little | Little | Slightly | Poor | Little |
| | poor | | | opaque | | |
| 1 | poor | Much | Much | Opaque | Bad | Much |

The results are shown in Table 4. The symbols are as follows.

TABLE 3

| | Average point |
|---|---|
| ++++ | at least 4.5 |
| +++ | from 3.5 to less than 4.5 |
| ++ | from 2.5 to less than 3.5 |
| + | from 1.5 to less than 2.5 |
| − | less than 1.5 |

TABLE 4

| | Example | Comparative Example | |
|---|---|---|---|
| Item | 1 | 1 | 2 |
| Spreading | ++++ | +++ | ++++ |
| Sticky feel | ++++ | +++ | +++ |
| Unevenness | ++++ | ++ | +++ |
| Transparency | ++++ | +++ | ++++ |
| Lasting quality | ++++ | ++ | + |
| Secondary sticking | +++ | + | + |

It is apparent from Table 4 that the cosmetic product according to the present invention is by far superior to those in Comparative Examples 1 and 2 in lasting quality and secondary sticking as well as usability.

The following formulations were used to prepare various cosmetic products according to the present invention. The samples were rated for “spreading” and “lasting quality” in the same way as mentioned above. The foundation was also rated for secondary sticking in the same way as mentioned above.

### Example 2

### Powder Foundation

| | Formulation | weight % |
|---|---|---|
| 1. | Silicone-treated titanium oxide (*1) | 12.0 |
| 2. | Silicone-treated sericite (*1) | 34.0 |
| 3. | Lecithin-treated talc | 35.1 |
| 4. | Lecithin-treated spherical nylon powder | 5.0 |
| 5. | Silicone-treated red oxide (*1) | 0.4 |
| 6. | Silicone-treated yellow iron oxide (*1) | 2.0 |
| 7. | Silicone-treated amber (*1) | 0.4 |
| 8. | Silicone-treated black iron oxide (*1) | 0.1 |
| 9. | Solution of polymer-A in D5 | 3.0 |
| 10. | Crosslinked dimethylsiloxane (*2) | 4.0 |
| 11. | Glyceryl trioctanoate | 1.5 |
| 12. | Silicone wax (*3) | 1.5 |

| | |
|---|---|
| Total | 100.0 |

*1: Treated with KF-9909 from Shin-Etsu Chemical Co., Ltd.
*2: KSG-16 from Shin-Etsu Chemical Co., Ltd.
*3: KP-562P from Shin-Etsu Chemical Co., Ltd.

### <Preparation of Cosmetic Product>

A: prepared from ingredients 1 to 8 by uniform mixing and crushing.
B: prepared from ingredients 9 to 12 by uniform mixing.
C: prepared by adding B to A, followed by uniform mixing.

The resulting mixture was press-formed in a mold to give the powder foundation as desired. The thus obtained powder foundation was found to be good in spreading and lasting quality and free of secondary sticking.

### Example 3

### Powder Foundation

| | Formulation | weight % |
|---|---|---|
| 1. | Caprylylsilane-treated mica (*1) | 40.0 |
| 2. | Silicone-treated talc (*2) | 24.6 |
| 3. | Silicone-treated titanium oxide (*2) | 10.0 |
| 4. | Silicone-treated fine powder of titanium oxide (*2) | 5.0 |
| 5. | Silicone-treated barium sulfate (*2) | 10.0 |
| 6. | Silicone-treated foundation pigment (*2) | q.s. |
| 7. | Phenyl-modified hybrid silicone composite powder (*3) | 2.0 |
| 8. | Polymethylsilsesquioxane powder (*4) | 0.4 |
| 9. | Antiseptic | q.s. |
| 10. | Perfume | q.s. |
| 11. | Solution of polymer-A in D5 | 4.0 |
| 12. | Glyceryl trioctanoate | 2.0 |
| 13. | Squalane | 1.0 |
| 14. | Vaseline | 1.0 |
| | Total | 100.0 |

*1: Treated with AES-3083 from Shin-Etsu Chemical Co., Ltd.
*2: Treated with KF-9909 from Shin-Etsu Chemical Co., Ltd.
*3: KSP-300 from Shin-Etsu Chemical Co., Ltd.
*4: KMP-590 from Shin-Etsu Chemical Co., Ltd.

### <Preparation of Cosmetic Product>

A: prepared from ingredients 1 to 9 by uniform mixing and crushing.
B: prepared from ingredients 11 to 14 by uniform mixing and added to A.
C: prepared adding ingredient 10 to B. The resulting mixture was press-formed in a mold to give the powder foundation as desired.

The thus obtained powder foundation was found to be good in spreading, feeling and lasting quality. Upon applied to skin, smooth, attractive and beautiful look was exerted.

### Example 4

### W/O Foundation in Stick Form

| | Formulation | weight % |
|---|---|---|
| 1. | Seresin | 5.5 |
| 2. | Inulin stearate | 2.0 |
| 3. | Neopentylglycol dioctanoate | 8.0 |
| 4. | Triethylhexanoin | 4.0 |
| 5. | Dimethylpolysiloxane (6 mm²/s at 25° C.) | 7.5 |
| 6. | Solution of polymer-A in D5 | 4.0 |
| 7. | Crosslinked polyglycerin-modified silicone (*1) | 4.0 |
| 8. | Alkyl-modified branched polyglycerin-modified | 1.5 |
| | silicone (*2) | |
| 9. | Spherical silicone resin powder (*3) | 1.5 |
| 10. | Silicone-treated titanium oxide (*4) | 9.0 |
| 11. | Silicone-treated foundation pigment (*5) | q.s. |
| 12. | Lecithin | 0.2 |
| 13. | Polysorbate 80 | 0.3 |
| 14. | 1,3-BG | 5.0 |
| 15. | Antiseptic | q.s. |
| 16. | Perfume | q.s. |
| 17. | Purified water | 47.5 |
| | Total | 100.0 |

*1: KSG-710 from Shin-Etsu Chemical Co., Ltd.
*2: KF-6105 from Shin-Etsu Chemical Co., Ltd.
*3: KMP-590 from Shin-Etsu Chemical Co., Ltd.
*4: treated with KF-9909 from Shin-Etsu Chemical Co., Ltd.
*5: treated with KF-9909 from Shin-Etsu Chemical Co., Ltd.

### <Preparation of Cosmetic Product>

A: prepared from ingredients 1 to 9 by uniform heating and dissolution.
B: prepared from ingredients 10 to 13 and a portion of ingredient 14 by rolling for dispersion.
C: prepared by uniformly dissolving the remainder of ingredient 14 and ingredients 15 and 17. C was added to B, followed by uniform dispersion with heating.
D: prepared by adding C to A for emulsification. After stirring with heating, ingredient 16 was added to D. The resulting product in stick form was filled into prescribed air-tight containers. Thus there was obtained the W/O foundation in stick form as desired.

The thus obtained W/O foundation was found to be good in spreading and lasting quality and free of secondary sticking.

### Example 5

### Makeup Remover

| | Formulation | weight % |
|---|---|---|
| 1. | POE (6) sorbitan monolaurate | 5.0 |
| 2. | Solution of polymer-B in TMF-1.5 | 1.0 |
| 3. | Polyglycerin-modified silicone (*1) | 15.0 |
| 4. | Ethanol | 10.0 |
| 5. | Dipropylene glycol | 3.0 |
| 6. | Glycerin | 2.0 |
| 7. | Sodium glutamate | 0.5 |
| 8. | Antiseptic | q.s. |
| 9. | Perfume | q.s. |
| 10. | Purified water | 63.5 |
| | Total | 100.0 |

*1: KF-6100 from Shin-Etsu Chemical Co., Ltd.

### <Preparation of Cosmetic Product>

A: prepared from ingredients 1 to 10 by uniform mixing. Thus there was obtained the makeup remover as desired.

The thus obtained makeup remover was used for removing a foundation. It was found to have good attachment effect to cosmetics and sebum stain, and good stain removal effect without sticky feel after use.

### Example 6

### Oil-in-Polyhydric Alcohol Emulsion Cream

| | Formulation | weight % |
|---|---|---|
| 1. | Crosslinked polyether-modified silicone (*1) | 3.0 |
| 2. | Crosslinked dimethylpolysiloxane (*2) | 27.0 |
| 3. | Branched polyether-modified silicone (*3) | 2.0 |
| 4. | Decamethylcyclopentasiloxane | 10.0 |
| 5. | Dimethylpolysiloxane (6 mm²/s at 25° C.) | 3.0 |
| 6. | Solution of polymer-A in D5 | 10.0 |
| 7. | Dimethyldistearylammonium hectorite | 2.0 |
| 8. | Antiseptic | q.s. |
| 9. | Perfume | q.s. |
| 10. | 1,3-butylene glycol | 43.0 |
| | Total | 100.0 |

*1: KSG-210 from Shin-Etsu Chemical Co., Ltd.
*2: KSG-15 from Shin-Etsu Chemical Co., Ltd.
*3: KF-6028 from Shin-Etsu Chemical Co., Ltd.

### <Preparation of Cosmetic Product>

A: prepared from ingredients 1 to 7 and 9 by uniform mixing.
B: prepared from ingredients 8 and 10 by mixing.
C: prepared by adding B to A, followed by uniform emulsification. There was obtained the cream as desired.

The oil-in-polyhydric alcohol emulsion cream obtained as mentioned above was found to be good in spreading and moist feel and free of sticky feel and oily feel, as well as excellent in lasting moisturizing effect.

### Example 7

### Oil-in-Polyhydric Alcohol Emulsion Cream

| | Formulation | weight % |
|---|---|---|
| 1. | Crosslinked polyglycerin-modified silicone (*1) | 5.0 |
| 2. | Crosslinked dimethylpolysiloxane (*2) | 30.0 |
| 3. | Crosslinked dimethylpolysiloxane (*3) | 35.0 |
| 4. | Branched polyglycerin-modified silicone (*4) | 1.0 |
| 5. | Decamethylcyclopentasiloxane | 5.0 |
| 6. | Solution of polymer-A in D5 | 4.0 |
| 7. | Phenyl-modified hybrid silicone composite powder (*5) | 5.0 |
| 8. | Antiseptic | q.s. |
| 9. | Perfume | q.s. |
| 10. | Glycerin | 5.0 |
| 11. | 1,3-butylene glycol | 10.0 |
| | Total | 100.0 |

*1: KSG-710 from Shin-Etsu Chemical Co., Ltd.
*2: KSG-15 from Shin-Etsu Chemical Co., Ltd.
*3: KSG-1610 from Shin-Etsu Chemical Co., Ltd.
*4: KF-6104 from Shin-Etsu Chemical Co., Ltd.
*5: KSP-300 from Shin-Etsu Chemical Co., Ltd.

### <Preparation of Cosmetic Product>

A: prepared from ingredients 1 to 7 and 9 by uniform mixing.
B: prepared from ingredients 8, 10, and 11 by mixing.
C: prepared by adding B to A, followed by uniform emulsification. There was obtained the cream as desired.

The oil-in-polyhydric alcohol emulsion cream obtained as mentioned above was found to be good in spreading and moist feel and free of sticky feel and oily feel, as well as excellent in lasting moisturizing effect.

### Example 8

### Blusher in Solid Form of Oil-in-Polyhydric Alcohol Emulsion Type

| | Formulation | weight % |
|---|---|---|
| 1. | Crosslinked polyglycerin-modified silicone (*1) | 5.0 |
| 2. | Crosslinked dimethylpolysiloxane (*2) | 5.0 |
| 3. | Decamethylcyclopentasiloxane | 3.0 |
| 4. | Dimethylpolysiloxane (6 cs) | 19.7 |
| 5. | Cetyl isooctanoate | 5.0 |
| 6. | Solution of polymer-A in D5 | 10.0 |
| 7. | Behenyl-modified acrylsilicone resin (*3) | 3.0 |
| 8. | Paraffin wax (m.p. 80° C.) | 9.0 |
| 9. | Dimethyldistearylammoniumhectorite | 0.3 |
| 10. | Acrylsilicone-treated powder (*4) | 25.0 |
| 11. | Antiseptic | q.s. |
| 12. | Perfume | q.s. |
| 13. | 1,3-butylene glycol | 15.0 |
| | Total | 100.0 |

*1: KSG-710 from Shin-Etsu Chemical Co., Ltd.
*2: KSG-15 from Shin-Etsu Chemical Co., Ltd.
*3: KP-562P from Shin-Etsu Chemical Co., Ltd.
*4: treated with KP-574 from Shin-Etsu Chemical Co., Ltd.

### <Preparation of Cosmetic Product>

A: prepared from ingredients 1 to 9 and 12 by uniform mixing with heating at 80° C.
B: prepared by adding ingredient 10 to A, followed by uniform dispersion.
C: prepared from ingredients 11 and 13 by mixing. After heating to 80° C., C was added to B for emulsification. The resulting emulsion was cast into a metal dish, followed by cooling. Thus there was obtained the blusher as desired.

The blusher in solid form of oil-in-polyhydric alcohol emulsion type obtained as mentioned above was found to be good in spreading and free of sticky feel and oily feel, and excellent in color development.

### Example 9

### Lip Color in Cream Form

| | Formulation | weight % |
|---|---|---|
| 1. | Dextrin palmitate/ethylhexanoate (*1) | 9.0 |
| 2. | Triethylhexanoin | 10.0 |
| 3. | Solution of polymer-B in D5 | 10.0 |
| 4. | Alkyl-modified crosslinked dimethyl-polysiloxane (*2) | 8.0 |
| 5. | Alkyl-modified branched polyglycerin-modified | 2.0 |
| | silicone (*3) | |
| 6. | Decamethylcyclopentasiloxane | 38.0 |
| 7. | 1,3-butylene glycol | 5.0 |
| 8. | Purified water | 18.0 |
| 9. | Colored pigment | q.s. |
| 10. | Mica | q.s. |
| 11. | Perfume | q.s. |
| | Total | 100.0 |

*1: Rheopearl TT from Chiba Flour Milling Co., Ltd.
*2: KSG-43 from Shin-Etsu Chemical Co., Ltd.
*3: KF-6105 from Shin-Etsu Chemical Co., Ltd.

### <Preparation of Cosmetic Product>

A: prepared from a portion of ingredients 1 and 2 and ingredients 3 to 6 by uniform mixing with heating.
B: prepared by adding ingredient 9 to the remainder of ingredient 2 by rolling for dispersion. B was added to A, followed by uniform mixing.
C: prepared from ingredients 7 and 8 by mixing with heating. C was added to B with heating, followed by emulsification.
D: prepared by adding ingredients 10 and 11 to C. Thus there was obtained the lip color in cream form as desired.

The lip color in cream form obtained as mentioned above was found to be good in spreading on lips and lasting quality and free of oily feel. It was good in feeling and excellent in luster as compared with the conventional ones.

### Example 10

### Eye Liner

| | Formulation | weight % |
|---|---|---|
| 1. | Methyl trimethicone (*1) | 5.0 |
| 2. | Polyether-modified silicone (*2) | 3.0 |
| 3. | Solution of polymer-A in M3T | 48.5 |
| 4. | Solution of silicone network resin (*3) | 15.0 |
| 5. | Dimethyldistearylammoniumhectorite | 3.0 |
| 6. | Silicone-modified black iron oxide (*4) | 10.0 |
| 7. | 1,3-butylene glycol | 5.0 |
| 8. | Sodium sulfate | 0.5 |
| 9. | Antiseptic | q.s. |
| 10. | Purified water | 10.0 |
| | Total | 100.0 |

*1: TMF-1.5 from Shin-Etsu Chemical Co., Ltd.
*2: KF-6017 from Shin-Etsu Chemical Co., Ltd.
*3: KF-7312T from Shin-Etsu Chemical Co., Ltd.
*4: treated with KF-9901 from Shin-Etsu Chemical Co., Ltd.

### <Preparation of Cosmetic Product>

A: prepared from ingredients 1 to 5 by mixing, and ingredient 6 was added to the mixture by uniform mixing for dispersion.
B: prepared from ingredients 7 to 10 by mixing.
C: prepared by adding B to A, followed by emulsification. Thus there was obtained the eye liner as desired.

The eye liner obtained as mentioned above was found to be easy to use with refreshed feel and very good in lasting quality.

### Example 11

### Mascara

| | Formulation | weight % |
|---|---|---|
| 1. | Solution of polymer-A in isododecane | 29.5 |
| 2. | Dextrin palmitate/ethylhexanoate (*1) | 3.0 |
| 3. | Seresin | 2.5 |
| 4. | Behenyl-modified acrylsilicone resin (*2) | 2.0 |
| 5. | Bees wax | 3.5 |
| 6. | Triethylhexanoin | 3.0 |
| 7. | Dimethyldistearylammoniumhectorite | 4.0 |
| 8. | Lecithin | 0.5 |
| 9. | Isododecane | 31.0 |
| 10. | Silicone-treated pigment (*3) | 5.0 |
| 11. | Silica | 3.0 |
| 12. | Talc | 12.0 |
| 13. | Branched polyether-modified silicone (*4) | 1.0 |
| | Total | 100.0 |

*1: Rheopearl TT from Chiba Flour Milling Co., Ltd.
*2: KP-562P from Shin-Etsu Chemical Co., Ltd.
*3: treated with KF-9909 from Shin-Etsu Chemical Co., Ltd.
*4: KF-6028P from Shin-Etsu Chemical Co., Ltd.

### <Preparation of Cosmetic Product>

A: prepared by adding ingredients 7 and 13 to ingredient 9, followed by uniform mixing with heating.
B: prepared by adding ingredients 1 to 6 and 8 to A, followed by uniform mixing.
C: prepared by adding ingredients 10 to 12 to B, followed by rolling for uniform mixing. Thus there was obtained the mascara as desired.

The mascara obtained as mentioned above was found to easily attach to eyelashes and be very good in spreading and lasting quality without sticky feel. It was also confirmed that the mascara was excellent in lasting curl retention effect of eyelashes.

### Example 12

### Cream Eye Shadow

| | Formulation | weight % |
|---|---|---|
| 1. | Decamethylcyclopentasiloxane | 15.0 |
| 2. | Dimethylpolysiloxane (6 mm²/s at 25° C.) | 4.0 |
| 3. | Solution of polymer-A in D5 | 5.0 |
| 4. | Branched polyether-modified silicone (*1) | 1.5 |
| 5. | Pigment treated with acrylsilicone resin (*2) | 16.0 |
| 6. | Sodium chloride | 2.0 |
| 7. | Propylene glycol | 8.0 |
| 8. | Antiseptic | q.s. |
| 9. | Purified water | 48.5 |
| | Total | 100.0 |

*1: KF-6028P from Shin-Etsu Chemical Co., Ltd.
*2: treated with KP-574 from Shin-Etsu Chemical Co., Ltd.

### <Preparation of Cosmetic Product>

A: prepared from ingredients 1 to 4 by mixing, and then ingredient 5 was added, followed by uniform mixing for dispersion.
B: prepared from ingredients 6 to 9 by mixing.
C: prepared by adding B to A. Thus there was obtained the cream eye shadow as desired.

The cream eye shadow obtained as mentioned above was found to spread easily and last long without oily feeling and powdery feeling.

### Example 13

### Cream Eye Shadow

| | Formulation | weight % |
|---|---|---|
| 1. | Solution of acrylsilicone resin (*1) | 3.0 |
| 2. | Stearyl-modified acrylsilicone resin (*2) | 2.0 |
| 3. | Branched polyether-modified silicone (*3) | 1.5 |
| 4. | Decamethylcyclopentasiloxane | 20.3 |
| 5. | Solution of polymer-A in D5 | 10.0 |
| 6. | Dimethyldistearylammoniumhectorite | 1.2 |
| 7. | Pigment treated with acrylsilicone resin (*4) | 20.0 |
| 8. | Spherical nylon powder | 3.0 |
| 9. | Talc | 4.0 |
| 10. | Ethanol | 5.0 |
| 11. | Purified water | 30.0 |
| | Total | 100.0 |

*1: KP-545 from Shin-Etsu Chemical Co., Ltd.
*2: KP-561P from Shin-Etsu Chemical Co., Ltd.
*3: KF-6028P from Shin-Etsu Chemical Co., Ltd.
*4: treated with KP-574 from Shin-Etsu Chemical Co., Ltd.

### <Preparation of Cosmetic Product>

A: prepared from ingredients 1 to 6 by mixing, and then ingredients 7 to 9 were added, followed by uniform mixing for dispersion.
B: prepared from ingredients 10 and 11 by mixing.
C: prepared by adding B to A. Thus there was obtained the cream eye shadow as desired.

The cream eye shadow obtained as mentioned above was found to spread easily, last long, and remain fresh without oily feeling and powdery feeling.

### Example 14

### Suncut Milky Lotion

| | Formulation | weight % |
|---|---|---|
| 1. | Crosslinked polyether-modified silicone (*1) | 3.0 |
| 2. | Crosslinked dimethylpolysiloxane (*2) | 2.0 |
| 3. | Branched polyether-modified silicone (*3) | 1.0 |
| 4. | Solution of polymer-A in D5 | 8.0 |
| 5. | Decamethylcyclopentasiloxane | 2.0 |
| 6. | Isotridecyl isonanoate | 4.0 |
| 7. | Dispersion of titanium oxide (*4) | 25.0 |
| 8. | Dispersion of zinc oxide (*5) | 35.0 |
| 9. | 1,3-butylene glycol | 2.0 |
| 10. | Sodium citrate | 0.2 |
| 11. | Sodium chloride | 0.5 |
| 12. | Purified water | 17.3 |
| | Total | 100.0 |

*1: KSG-210 from Shin-Etsu Chemical Co., Ltd.
*2: KSG-15 from Shin-Etsu Chemical Co., Ltd.
*3: KF-6028P from Shin-Etsu Chemical Co., Ltd.
*4: SPD-T5 from Shin-Etsu Chemical Co., Ltd.
*5: SPD-Z5 from Shin-Etsu Chemical Co., Ltd.

### <Preparation of Cosmetic Product>

A: prepared from ingredients 1 to 6 by mixing.
B: prepared from ingredients 9 to 12 by mixing.
C: prepared by adding B to A, followed by emulsification, and then ingredients 7 and 8 were added. Thus there was obtained the suncut milky lotion as desired.

The suncut milky lotion obtained as mentioned above was found to spread easily, and exhibit good feeling without sticky feeling and oily feeling. It was also excellent in dispersibility of titanium oxide particles and zinc oxide particles and stability with the passage of time without whitening the skin. Moreover, it was also good in water resistance and sweat resistance and in sunburn-restraining effect.

### Example 15

### Suncut Cream

| | Formulation | weight % |
|---|---|---|
| 1. | Crosslinked polyether-modified silicone (*1) | 3.0 |
| 2. | Crosslinked dimethylpolysiloxane (*2) | 2.0 |
| 3. | Alkyl-modified branched polyether-modified silicone (*3) | 1.0 |
| 4. | Solution of polymer-A in D5 | 5.0 |
| 5. | Decamethylcyclopentasiloxane | 17.5 |
| 6. | Octyl methoxycinnamate | 6.0 |
| 7. | Solution of acrylsilicone resin (*4) | 10.0 |
| 8. | Lipophilized fine powder of zinc oxide (*5) | 20.0 |
| 9. | 1,3-butylene glycol | 2.0 |
| 10. | Sodium citrate | 0.2 |
| 11. | Sodium chloride | 0.5 |
| 12. | Perfume | q.s. |
| 13. | Purified water | 32.8 |
| | Total | 100.0 |

*1: KSG-240 from Shin-Etsu Chemical Co., Ltd.
*2: KSG-15 from Shin-Etsu Chemical Co., Ltd.
*3: KF-6038 from Shin-Etsu Chemical Co., Ltd.
*4: KP-575 from Shin-Etsu Chemical Co., Ltd.
*5: treated with AES-3083 from Shin-Etsu Chemical Co., Ltd.

### <Preparation of Cosmetic Product>

A: prepared by adding ingredient 7 to a portion of ingredient 5 and adding ingredient 8, followed by dispersion in a beads mill.
B: prepared from ingredients 1 to 4, the remainder of ingredient 5, and ingredient 6 by uniform mixing.
C: prepared from ingredients 9 to 11 and 13 by uniform mixing.
D: prepared by adding C to B, followed by emulsification, and then A and ingredient 12 were added. Thus there was obtained the suncut cream as desired.

The suncut cream obtained as mentioned above was found to spread easily, last long, and exhibit good refreshed feeling without sticky feeling and oily feeling.

### Example 16

### Suncut Lotion

### Shaking Type

| | Formulation | weight % |
|---|---|---|
| 1. | Branched polyether-modified silicone(*1) | 2.0 |
| 2. | Solution of polymer-A in D5 | 5.0 |
| 3. | Dimethylpolysiloxane (6 mm²/s at 25° C.) | 3.0 |
| 4. | Decamethylcyclopentasiloxane | 7.8 |
| 5. | Ethylhexyl methoxycinnamate | 7.5 |
| 6. | Hybrid silicone composite powder (*2) | 0.5 |
| 7. | Dimethyldistearylammoniumhectorite | 0.2 |
| 8. | Zinc oxide dispersion (*3) | 45.0 |
| 9. | 1,3-butylene glycol | 3.0 |
| 10. | Alcohol | 5.0 |
| 11. | Sodium citrate | 0.2 |
| 12. | Sodium chloride | 0.5 |
| 13. | Purified water | 20.3 |
| | Total | 100.0 |

*1: KF-6028P from Shin-Etsu Chemical Co., Ltd.
*2: KSP-105 from Shin-Etsu Chemical Co., Ltd.
*3: SPD-Z6 from Shin-Etsu Chemical Co., Ltd.

### <Preparation of Cosmetic Product>

A: prepared from ingredients 1 to 7 by uniform mixing.
B: prepared from ingredients 9 to 13 by mixing.
C: prepared by adding B to A, followed by emulsification, and then ingredient 8 was added. Thus there was obtained the suncut lotion of shaking type as desired.

The suncut lotion obtained as mentioned above was found to spread easily and last long without sticky feeling and oily feeling.

### Example 17

### Suncut Cream

| | Formulation | weight % |
|---|---|---|
| 1. | Crosslinked polyether-modified silicone (*1) | 2.0 |
| 2. | Crosslinked dimethylpolysiloxane (*2) | 3.0 |
| 3. | Branched polyether-modified silicone (*3) | 1.5 |
| 4. | Solution of polymer-A in D5 | 3.5 |
| 5. | Decamethylcyclopentasiloxane | 6.8 |
| 6. | Dimethyldistearylammoniumhectorite | 1.2 |
| 7. | Titanium oxide dispersion (*4) | 20.0 |
| 8. | Zinc oxide dispersion (*5) | 15.0 |
| 9. | 1,3-butylene glycol | 5.0 |
| 10. | Sodium citrate | 0.2 |
| 11. | Sodium chloride | 0.5 |
| 12. | Purified water | 41.3 |
| | Total | 100.0 |

*1: KSG-210 from Shin-Etsu Chemical Co., Ltd.
*2: KSG-15 from Shin-Etsu Chemical Co., Ltd.
*3: KF-6028P from Shin-Etsu Chemical Co., Ltd.
*4: SPD-T6 from Shin-Etsu Chemical Co., Ltd.
*5: SPD-Z6 from Shin-Etsu Chemical Co., Ltd.

### <Preparation of Cosmetic Product>

A: prepared from ingredients 1 to 8 by uniform mixing.
B: prepared from ingredients 9 to 12 by mixing.
C: prepared by adding B to A. Thus there was obtained the suncut cream as desired.

The suncut cream obtained as mentioned above was found to spread easily and last long without sticky feeling and oily feeling.

### Example 18

### Suntan Milky Lotion

| | Formulation | weight % |
|---|---|---|
| 1. | Crosslinked polyether-modified silicone (*1) | 2.0 |
| 2. | Crosslinked dimethylpolysiloxane (*2) | 3.0 |
| 3. | Polyether-modified silicone (*3) | 1.5 |
| 4. | Solution of polymer-A in D5 | 10.0 |
| 5. | Dimethylpolysiloxane (6 mm²/s at 25° C.) | 10.0 |
| 6. | Decamethylcyclopentasiloxane | 16.5 |
| 7. | Dihydroxyacetone | 2.0 |
| 8. | Glycerin | 8.0 |
| 9. | 1, 3-butylene glycol | 5.0 |
| 10. | Sodium citrate | 0.2 |
| 11. | Sodium chloride | 0.5 |
| 12. | Antioxidant | q.s. |
| 13. | Antiseptic | q.s. |
| 14. | Perfume | q.s. |
| 15. | Purified water | 41.3 |
| | Total | 100.0 |

*1: KSG-210 from Shin-Etsu Chemical Co., Ltd.
*2: KSG-15 from Shin-Etsu Chemical Co., Ltd.
*3: KF-6017 from Shin-Etsu Chemical Co., Ltd.

### <Preparation of Cosmetic Product>

A: prepared from ingredients 1 to 6 by uniform mixing.
B: prepared from ingredients 7 to 13 and 15 by mixing.
C: prepared by adding B to A, followed by addition of ingredient 14. Thus there was obtained the suntan cream as desired.

The suntan cream obtained as mentioned above was found to spread easily and give light feeling without sticky feeling and oily feeling.

### Example 19

### Suntan Cream

| | Formulation | weight % |
|---|---|---|
| 1. | Alkyl-modified crosslinked polyether-modified silicone (*1) | 4.0 |
| 2. | Alkyl-modified crosslinked dimethylpoly-siloxane (*2) | 2.0 |
| 3. | Alkyl-modified branched polyether-modified silicone (*3) | 1.0 |
| 4. | Solution of polymer-A in D5 | 5.0 |
| 5. | Decamethylcyclopentasiloxane | 10.5 |
| 6. | Stearyl-modified acrylsilicone (*4) | 1.0 |
| 7. | Dimethyloctylparaaminobenzoic acid | 1.5 |
| 8. | 4-t-butyl-4′-methoxy-dibenzoylmethane | 1.5 |
| 9. | Kaolin | 0.5 |
| 10. | Pigment | 8.0 |
| 11. | Titanium oxide-coated mica | 8.0 |
| 12. | Dioctadecyldimethylammonium chloride | 0.1 |
| 13. | Sodium L-glutamate | 3.0 |
| 14. | 1, 3-butylene glycol | 5.0 |
| 15. | Sodium citrate | 0.2 |
| 16. | Sodium chloride | 0.5 |
| 17. | Antioxidant | q.s. |
| 18. | Antiseptic | q.s. |
| 19. | Perfume | q.s. |
| 20. | Purified water | 48.2 |
| | Total | 100.0 |

*1: KSG-320 from Shin-Etsu Chemical Co., Ltd.
*2: KSG-42 from Shin-Etsu Chemical Co., Ltd.
*3: KF-6038 from Shin-Etsu Chemical Co., Ltd.
*4: KP-561P from Shin-Etsu Chemical Co., Ltd.

### <Preparation of Cosmetic Product>

A: prepared from ingredients 1 to 8, 17, and 18 by mixing with heating.
B: prepared from ingredients 12 and a portion of ingredient 20 by mixing with heating, and then ingredients 9 to 11 were added, followed by dispersion.
C: prepared from ingredients 13 to 16 and the remainder of ingredient 20 by uniform dissolution. Then, C was mixed with B.
D: prepared by slowly adding C to A with stirring for emulsification. After cooling, ingredient 19 was added. Thus there was obtained the suntan cream as desired.

The suntan cream obtained as mentioned above was found to have fine texture, spread easily, last long, and give light feeling without sticky feeling and oily feeling.

### Example 20

### Liquid W/O Foundation

| | Formulation | weight % |
|---|---|---|
| 1. | Decamethylcyclopentasiloxane | 15.0 |
| 2. | Dimethylpolysiloxane (6 mm²/s at 25° C.) | 2.0 |
| 3. | Solution of polymer-A in D5 | 5.0 |
| 4. | Solution of polymer-B in M3T | 5.0 |
| 5. | Alkyl-modified branched polyether-modified silicone (*1) | 2.0 |
| 6. | Ethylhexyl paramethoxycinnamate | 3.0 |
| 7. | Fluorine-modified silicone (*2) | 2.0 |
| 8. | Spherical silicone resin powder (*3) | 1.5 |
| 9. | Foundation pigment treated with fluorine compound (*4) | |
| 10. | Fine particles of mica titanium treated with fluorine | 2.0 |
| | compound (*4) | |
| 11. | Fine particles of titanium oxide treated with | 8.0 |
| | silicone (*5) | |
| 12. | Alkyl-modified branched polyglycerin-modified silicone (*6) | 1.2 |
| 13. | Ethanol | 3.0 |
| 14. | 1, 3-butylene glycol | 5.0 |
| 15. | Glycerin | 2.0 |
| 16. | Magnesium sulfate | 0.5 |
| 17. | Antioxidant | q.s. |
| 18. | Antiseptic | q.s. |
| 19. | Perfume | q.s. |
| 20. | Purified water | 33.5 |
| | Total | 100.0 |

*1: KF-6038 from Shin-Etsu Chemical Co., Ltd.
*2: FL-5 from Shin-Etsu Chemical Co., Ltd.
*3: KMP-590 from Shin-Etsu Chemical Co., Ltd.
*4: coated with diethanolamine salt of perfluoroalkyl-ethylphosphate (5%)
*5: treated with KF-9909 from Shin-Etsu Chemical Co., Ltd.
*6: KF-6105 from Shin-Etsu Chemical Co., Ltd.

### <Preparation of Cosmetic Product>

A: prepared from a portion of ingredient 1, ingredients 11 and 12, and a portion of ingredient 20 by mixing, followed by uniform dispersion.
B: prepared from ingredients 8 to 10 by uniform mixing.
C: prepared from the remainder of ingredient 1 and ingredients 2 to 7 by mixing. C was added to B for uniform dispersion and mixing.
D: prepared from ingredients 13 to 18 and the remainder of ingredient 20 by uniform mixing.
E: D was slowly added to C with stirring for emulsification, and A and ingredient 19 were added. Thus there was obtained the W/O foundation as desired.

The W/O foundation obtained as mentioned above was found to spread easily, last long, and give light feeling without sticky feeling and oily feeling. The pigment was difficult to settle out in the liquid foundation. Thus, the foundation was excellent in stability.

### Example 21

### Hair Cream

| | Formulation | weight % |
|---|---|---|
| 1. | Decamethylcyclopentasiloxane | 16.0 |
| 2. | Methylphenylpolysiloxane (*1) | 2.0 |
| 3. | Solution of polymer-A in D5 | 4.0 |
| 4. | Squalane | 5.0 |
| 5. | Solution of silicone network resin (*2) | 2.0 |
| 6. | Sorbitan sesquiisostearate | 1.5 |
| 7. | Alkyl-modified branched polyether-modified silicone (*3) | 2.0 |
| 8. | Sodium sorbitol sulfate | 2.0 |
| 9. | Sodium chondroitin sulfate | 1.0 |
| 10. | Sodium hyaluronate | 0.5 |
| 11. | Propylene glycol | 3.0 |
| 12. | Antiseptic | 1.5 |
| 13. | Vitamin E acetate | 0.1 |
| 14. | Antioxidant | q.s. |
| 15. | Perfume | q.s. |
| 16. | Purified water | 59.4 |
| | Total | 100.0 |

*1: KF-54 from Shin-Etsu Chemical Co., Ltd.
*2: KF-7312J from Shin-Etsu Chemical Co., Ltd.
*3: KF-6038 from Shin-Etsu Chemical Co., Ltd.

### <Preparation of Cosmetic Product>

A: prepared from ingredients 1 to 7 and 12 to 14 by uniform mixing.
B: prepared from ingredients 8 to 11 and 16 by uniform mixing.
C: prepared by slowly adding B to A with stirring for emulsification, and then ingredient 15 was added. Thus there was obtained the hair cream as desired.

The hair cream obtained as mentioned above was found to give easy application to hair, exhibit smooth and dry feeling without oily feeling and good hair style retention, and impart good luster to hair.

### Example 22

### Hair Cream

| | Formulation | weight % |
|---|---|---|
| 1. | Solution of silicone gum (*1) | 10.0 |
| 2. | Solution of silicone network resin (*2) | 10.0 |
| 3. | Solution of polymer-A in M3T | 10.0 |
| 4. | Glyceryl tri-2-ethylhexanoate | 5.0 |
| 5. | Vaseline | 5.0 |
| 6. | Stearic acid | 1.5 |
| 7. | Cetyl alcohol | 0.5 |
| 8. | Polyglyceryl monooleate | 1.5 |
| 9. | Glyceryl monostearate | 1.5 |
| 10. | Polyether-modified silicone (*3) | 0.5 |
| 11. | 1, 3-butylene glycol | 5.0 |
| 12. | Acrylates/alkyl acrylate (C10-30) cross-polymer (*4) | 0.3 |
| 13. | Triethanolamine | 0.3 |
| 14. | Antiseptic | q.s. |
| 15. | Perfume | q.s. |
| 16. | Purified water | 48.9 |
| | Total | 100.0 |

*1: KF-9028 from Shin-Etsu Chemical Co., Ltd.
*2: KF-7312J from Shin-Etsu Chemical Co., Ltd.
*3: KF-6011 from Shin-Etsu Chemical Co., Ltd.
*4: Pemulen TR-1 from Noveon Inc.

### <Preparation of Cosmetic Product>

A: prepared from ingredients 1 to 10 and 14 by heating for dissolution.
B: prepared from ingredients 11 to 13 and 16 by mixing with heating.
C: prepared by adding B to A with stirring for emulsification. After cooling, ingredient 15 was added. Thus there was obtained the hair cream as desired.

The hair cream obtained as mentioned above was found to spread easily, last long, impart gloss and smoothness to hair, produce good hair setting effect.

### Example 23

### Moisturizing O/W Cream

| | Formulation | weight % |
|---|---|---|
| 1. | Decamethylcyclopentasiloxane | 9.0 |
| 2. | Solution of polymer-A in D5 | 4.0 |
| 3. | Liquid paraffin | 5.0 |
| 4. | Stearoxy-modified silicone (*1) | 8.0 |
| 5. | Polyether-modified silicone (*2) | 2.0 |
| 6. | Polymethylsilsesquioxane spherical powder (*3) | 2.5 |
| 7. | Hydrophobic silica | 2.0 |
| 8. | Zinc stearate | 2.0 |
| 9. | Vitamin E acetate | 3.0 |
| 10. | Polyacrylamide (*4) | 0.5 |
| 11. | Polyethylene glycol 400 | 1.0 |
| 12. | Sodium lactate | 1.0 |
| 13. | 1, 3-butylene glycol | 5.0 |
| 14. | Antiseptic | q.s. |
| 15. | Perfume | q.s. |
| 16. | Purified water | 55.0 |
| | Total | 100.0 |

*1: KF-7002 from Shin-Etsu Chemical Co., Ltd.
*2: KF-6011P from Shin-Etsu Chemical Co., Ltd.
*3: KSP-100 from Shin-Etsu Chemical Co., Ltd.
*4: Sepigel 305 from Sepik Co., Ltd.

### <Preparation of Cosmetic Product>

A: prepared from ingredients 1 to 9 and 14 by uniform mixing.
B: prepared from ingredients 10 to 13 and 16 by uniform mixing.
C: prepared by adding B to A with stirring for emulsification, then ingredient 15 was added. Thus there was obtained the moisturizing O/W cream as desired.

The moisturizing O/W cream obtained as mentioned above was found to spread easily, impart refreshing feeling, and keep long the moisturizing effect.

### Example 24

### O/W Emollient Cream

| | Formulation | weight % |
|---|---|---|
| 1. | Crosslinked dimethylpolysiloxane (*1) | 7.0 |
| 2. | Crosslinked dimethylpolysiloxane (*2) | 30.0 |
| 3. | Solution of polymer-A in D5 | 6.0 |
| 4. | Decamethylcyclopentasiloxane | 5.0 |
| 5. | 1, 3-butylene glycol | 4.0 |
| 6. | Branched polyglycerin-modified silicone (*3) | 0.6 |
| 7. | Branched polyglycerin-modified silicone (*4) | 0.3 |
| 8. | (Acrylamide/acryloyldimethyltaulin Na) copolymer (*5) | 0.6 |
| 9. | Dimethyltaulinammonium acrylate/VP copolymer (*6) | 0.7 |
| 10. | Sodium chloride | 0.1 |
| 11. | Purified water | 45.7 |
| | Total | 100.0 |

*1: KSG-15 from Shin-Etsu Chemical Co., Ltd.
*2: KSG-16 from Shin-Etsu Chemical Co., Ltd.
*3: KF-6104 from Shin-Etsu Chemical Co., Ltd.
*4: KF-6100 from Shin-Etsu Chemical Co., Ltd.
*5: Simulgel 600 from Sepik Co., Ltd.
*6: Aristoflex AVC from Clariant Inc.

### <Preparation of Cosmetic Product>

A: prepared from ingredients 1 to 4 by uniform mixing.
B: prepared from ingredients 5 to 11 by uniform mixing.
C: prepared by adding B to A with stirring. Thus there was obtained the O/W emollient cream as desired.

The O/W emollient cream obtained as mentioned above was found to spread easily, impart dry feeling, and effectively protect the skin without oily feeling.

### Example 25

### Hand Cream

| | Formulation | weight % |
|---|---|---|
| 1. | Decamethylcyclopentasiloxane | 25.0 |
| 2. | Solution of polymer-A in D5 | 10.0 |
| 3. | Liquid paraffin | 5.0 |
| 4. | Solution of amino-modified silicone gum (*1) | 8.0 |
| 5. | Branched polyether-modified silicone (*2) | 2.0 |
| 6. | Hybrid silicone composite powder (*3) | 2.5 |
| 7. | Distearyldimethylammonium chloride | 0.8 |
| 8. | Vitamin E acetate | 0.1 |
| 9. | Polyethylene glycol 400 | 1.0 |
| 10. | Glycerin | 10.0 |
| 11. | Aluminum magnesium silicate | 1.2 |
| 12. | Antiseptic | q.s. |
| 13. | Perfume | q.s. |
| 14. | Purified water | 34.4 |
| | Total | 100.0 |

*1: KF-8108 from Shin-Etsu Chemical Co., Ltd.
*2: KF-6028P from Shin-Etsu Chemical Co., Ltd.
*3: KSP-102 from Shin-Etsu Chemical Co., Ltd.

### <Preparation of Cosmetic Product>

A: prepared from ingredients 1 to 8 and 12 by uniform mixing.
B: prepared from ingredients 9 to 11 and 14 by uniform mixing.
C: prepared by adding B to A with stirring for emulsification, and then ingredient 13 was added. Thus there was obtained the hand cream as desired.

The hand cream obtained as mentioned above was found to spread easily, impart dry feeling, and effectively protect the skin without oily feeling.

### Example 26

### O/W Cream

| | Formulation | weight % |
|---|---|---|
| 1. | Dimethylpolysiloxane (6 mm²/s at 25° C.) | 5.0 |
| 2. | Stearyl-modified acrylsilicone resin (*1) | 8.0 |
| 3. | Solution of polymer-A in D5 | 10.0 |
| 4. | Glyceryl triisostearate | 8.0 |
| 5. | Cetanol | 1.0 |
| 6. | Stearic acid | 3.0 |
| 7. | Glyceryl monostearate | 1.5 |
| 8. | Sorbitan sesquioleate | 0.5 |
| 9. | Polyoxyethylenesorbitan monooleate | 1.0 |
| 10. | Sodium hydroxide (1 wt % aqueous solution) | 10.0 |
| 11. | 1, 3-butylene glycol | 5.0 |
| 12. | Antiseptic | q.s. |
| 13. | Perfume | q.s. |
| 14. | Purified water | 54.3 |
| | Total | 100.0 |

*1: KP-561P from Shin-Etsu Chemical Co., Ltd.

### <Preparation of Cosmetic Product>

A: prepared from ingredients 1 to 9 by mixing with heating.
B: prepared from ingredients 10 to 12 and 14 by mixing with heating.
C: prepared by slowly adding B to A with stirring for emulsification. After cooling, ingredient 13 was added. Thus there was obtained the O/W cream as desired.

The O/W cream obtained as mentioned above was found to spread easily and impart dry feeling without sticky feeling and oily feeling.

### Example 27

### O/W Cream

| | Formulation | weight % |
|---|---|---|
| 1. | Polyglyceryl monooleate | 1.0 |
| 2. | Cetyl alcohol | 0.5 |
| 3. | Stearic acid | 1.0 |
| 4. | Glyceryl monostearate | 1.0 |
| 5. | Solution of polymer-A in D5 | 1.0 |
| 6. | Macademianut oil | 10.0 |
| 7. | Crosslinked dimethylpolysiloxane (*1) | 0.5 |
| 8. | Acrylates/alkyl acrylate (C10-30) cross-polymer (*2) | 0.2 |
| 9. | Methyl cellulose | 0.1 |
| 10. | Triethanolamine | 0.2 |
| 11. | 1, 3-butylene glycol | 7.0 |
| 12. | Antiseptic | q.s. |
| 13. | Perfume | q.s. |
| 14. | Purified water | 77.5 |
| | Total | 100.0 |

*1: KSG-16 from Shin-Etsu Chemical Co., Ltd.
*2: Pemulen TR-1 from Noveon Inc.

### <Preparation of Cosmetic Product>

A: prepared from ingredients 1 to 7 by mixing with heating.
B: prepared from ingredients 8 to 12 and 14 by mixing with heating.
C: prepared by slowly adding B to A with stirring for emulsification. After cooling, ingredient 13 was added. Thus there was obtained the O/W cream as desired.

The O/W cream obtained as mentioned above was found to spread easily, impart dry feeling, and keep the skin fresh without sticky feeling and oily feeling.

### Example 28

### Transparent W/O Cosmetic Solution

| | Formulation | weight % |
|---|---|---|
| 1. | Crosslinked polyether-modified silicone (*1) | 3.0 |
| 2. | Crosslinked dimethylpolysiloxane (*2) | 40.0 |
| 3. | Crosslinked dimethylpolysiloxane (*3) | 25.0 |
| 4. | Solution of polymer-A in D5 | 10.0 |
| 5. | Polyether-modified silicone (*4) | 1.5 |
| 6. | Dimethylpolysiloxane (6 mm²/s at 25° C.) | 2.0 |
| 7. | Decamethylcyclopentasiloxane | 5.3 |
| 8. | Glycerin | 4.5 |
| 9. | 1, 3-butylene glycol | 2.0 |
| 10. | Sodium citrate | 0.2 |
| 11. | Sodium chloride | 0.5 |
| 12. | Purified water | 6.0 |
| | Total | 100.0 |

*1: KSG-210 from Shin-Etsu Chemical Co., Ltd.
*2: KSG-15 from Shin-Etsu Chemical Co., Ltd.
*3: KSG-1610 from Shin-Etsu Chemical Co., Ltd.
*4: KF-6017 from Shin-Etsu Chemical Co., Ltd.

### <Preparation of Cosmetic Product>

A: prepared from ingredients 1 to 7 by uniform mixing.
B: prepared from ingredients 8 to 12 by uniform mixing.
C: prepared by slowly adding B to A with stirring for emulsification. Thus there was obtained the transparent W/O cosmetic solution as desired.

The transparent W/O cosmetic solution obtained as mentioned above was found to keep the skin fresh without sticky feeling and oily feeling.

### Example 29

### Antiperspirant

| | Formulation | weight % |
|---|---|---|
| 1. | Crosslinked polyether-modified silicone (*1) | 7.0 |
| 2. | Solution of polymer-A in D5 | 8.0 |
| 3. | Decamethylcyclopentasiloxane | 9.0 |
| 4. | 1, 3-butylene glycol | 5.0 |
| 5. | Sodium citrate | 0.2 |
| 6. | Glycine salt of aluminum zirconium tetra-chloride hydrate | 20.0 |
| 7. | Purified water | 50.8 |
| | Total | 100.0 |

*1: KSG-210 from Shin-Etsu Chemical Co., Ltd.

### <Preparation of Cosmetic Product>

A: prepared from ingredients 1 to 3 by uniform mixing.
B: prepared from ingredients 4 to 7 by uniform mixing.
C: prepared by slowly adding B to A with stirring for emulsification. Thus there was obtained the antiperspirant as desired.

The antiperspirant obtained as mentioned above was found to spread easily, last long, and produce antiperspirant effect without whitening the skin.

### Example 30

### Wrinkle Concealer

| | Formulation | weight % |
|---|---|---|
| 1. | Crosslinked polyether-modified silicone (*1) | 5.0 |
| 2. | Crosslinked dimethylpolysiloxane (*2) | 55.0 |
| 3. | Solution of polymer-A in D5 | 15.0 |
| 4. | Decamethylcyclopentasiloxane | 8.0 |
| 5. | Hybrid silicone composite powder (*3) | 12.0 |
| 6. | Silicone gum solution (*4) | 5.0 |
| | Total | 100.0 |

*1: KSG-210 from Shin-Etsu Chemical Co., Ltd.
*2: KSG-15 from Shin-Etsu Chemical Co., Ltd.
*3: KSP-101 from Shin-Etsu Chemical Co., Ltd.
*4: KF-9028 from Shin-Etsu Chemical Co., Ltd.

### <Preparation of Cosmetic Product>

A: prepared from ingredients 1 to 6 by uniform mixing. Thus there was obtained the wrinkle concealer as desired.

The wrinkle concealer obtained as mentioned above was found to spread easily, impart light feeling, and keep long the concealing effect without sticky feeling and oily feeling.

### Example 31

### Cleansing Cream

| | Formulation | weight % |
|---|---|---|
| 1. | Dimethylpolysiloxane (6 mm²/s at 25° C.) | 5.0 |
| 2. | Methylphenylpolysiloxane (*1) | 5.0 |
| 3. | Liquid paraffin | 5.0 |
| 4. | Jojoba oil | 2.0 |
| 5. | Solution of polymer-A in D5 | 4.0 |
| 6. | Branched polyether-modified silicone (*2) | 2.0 |
| 7. | Dextrin fatty acid ester | 0.8 |
| 8. | Aluminum monostearate | 0.2 |
| 9. | Aluminum chloride | 1.0 |
| 10. | Glycerin | 10.0 |
| 11. | Antiseptic | q.s. |
| 12. | Perfume | q.s. |
| 13. | Purified water | 65.0 |
| | Total | 100.0 |

*1: KF-56 from Shin-Etsu Chemical Co., Ltd.
*2: KF-6028 from Shin-Etsu Chemical Co., Ltd.

### <Preparation of Cosmetic Product>

A: prepared from ingredients 1 to 8 by mixing with heating.
B: prepared from ingredients 9 to 11 and 13 by mixing with heating.
C: prepared by slowly adding B to A with stirring for emulsification. After cooling, ingredient 12 was added. Thus there was obtained the cleansing cream as desired.

The cleansing cream obtained as mentioned above was found to spread easily and impart refresh feeling, moist feeling, and light feeling.

### Example 32

### Transparent Cleansing Lotion

| | Formulation | weight % |
|---|---|---|
| 1. | Decamethylcyclopentasiloxane | 50.0 |
| 2. | Solution of polymer-A in D5 | 5.8 |
| 3. | Neopentylglycol dioctanoate | 6.0 |
| 4. | Silica | 0.2 |
| 5. | 1, 3-butylene glycol | 5.0 |
| 6. | Glycerin | 6.0 |
| 7. | Polyether-modified silicone (*1) | 5.0 |
| 8. | Polyether-modified silicone (*2) | 3.0 |
| 9. | PEG-60 hydrogenated castor oil | 2.0 |
| 10. | Purified water | 17.0 |
| | Total | 100.0 |

*1: KF-6011 from Shin-Etsu Chemical Co., Ltd.
*2: KF-6013 from Shin-Etsu Chemical Co., Ltd.

### <Preparation of Cosmetic Product>

A: prepared from ingredients 1 to 4 by uniform mixing.
B: prepared from ingredients 5 to 10 by uniform mixing.
C: prepared by slowly adding B to A with stirring for emulsification. Thus there was obtained the transparent cleansing lotion as desired.

The transparent cleansing lotion obtained as mentioned above was found to spread easily and impart refresh feeling and moist feeling, and produce good cleansing effect.

### Example 33

### Body Lotion

| | Formulation | weight % |
|---|---|---|
| 1. | Alcohol | 17.0 |
| 2. | 1,3-butyrene glycol | 3.0 |
| 3. | Branched polyglycerin-modified silicone (*1) | 0.5 |
| 4. | Polymer-C powder | 1.0 |
| 5. | Triethylhexanoin | 1.0 |
| 6. | Hybrid silicone composite powder (*2) | 10.0 |
| 7. | Dimethyltaulinammonium acrylate/VP copolymer (*3) | 0.4 |
| 8. | Xanthane gum (2 weight % solution) | 6.0 |
| 9. | Sodium chloride | 0.1 |
| 10. | Purified water | 61.0 |
| | Total | 100.0 |

*1: KF-6100 from Shin-Etsu Chemical Co., Ltd.
*2: KSP-100 from Shin-Etsu Chemical Co., Ltd.
*3: Aristoflex manufactured by Clariant International Ltd.

### <Preparation of Cosmetic Product>

A: prepared from ingredients 1 to 6 by uniform mixing.
B: prepared from ingredients 7 to 10 by uniform mixing.
C: prepared by slowly adding B to A and mixing them. Thus there was obtained the body lotion as desired.

The body lotion obtained as mentioned above was found to impart dry feeling without sticky feeling and oily feeling, spread easily, and keep refresh feeling.

### Example 34

### Treatment Gel

| | Formulation | weight % |
|---|---|---|
| 1. | Alcohol | 20.0 |
| 2. | Polymer-C powder | 1.5 |
| 3. | Triethylhexanoin | 1.5 |
| 4. | Polyether-modified silicone (*1) | 0.5 |
| 5. | Stearyl-modified acrylsilicone resin (*2) | 1.0 |
| 6. | Phenyl-modified hybrid silicone composite powder (*3) | 8.0 |
| 7. | Carboxyvinyl polymer | 0.2 |
| 8. | Triethanolamine | 0.2 |
| 9. | Antiseptic | q.s. |
| 10. | Purified water | 67.1 |
| | Total | 100.0 |

*1: KF-6011 from Shin-Etsu Chemical Co., Ltd.
*2: KP-561P from Shin-Etsu Chemical Co., Ltd.
*3: KSP-300 from Shin-Etsu Chemical Co., Ltd.

### <Preparation of Cosmetic Product>

A: prepared from ingredients 1 to 6 by uniform mixing under heating.
B: prepared from ingredients 7 to 10 by uniform mixing.
C: prepared by adding A to B with stirring. Thus there was obtained the treatment gel as desired.

The treatment gel obtained as mentioned above was found to have smooth and dry feeling without oily feeling, spread easily, and impart excellent treatment effect.

### Example 35

### Rinse Type Face Pack

| | Formulation | weight % |
|---|---|---|
| 1. | Dimethylpolysiloxane (6 mm²/s at 25° C.) | 3.0 |
| 2. | Solution of polymer-A in D5 | 2.0 |
| 3. | Polyether-modified silicone (*1) | 0.5 |
| 4. | Kaolin | 30.0 |
| 5. | Carboxyvinyl polymer | 4.0 |
| 6. | 1,3-butyrene glycol | 10.0 |
| 7. | Glycerin | 20.0 |
| 8. | Triethanolamine | 0.4 |
| 9. | Antiseptic | q.s. |
| 10. | Purified water | 0.3 |
| | Total | 100.0 |

*1: KF-6011 from Shin-Etsu Chemical Co., Ltd.

### <Preparation of Cosmetic Product>

A: prepared from ingredients 1 to 3 by uniform mixing.
B: prepared from ingredients 5 to 10 by uniform mixing, followed by adding ingredient 4 thereto and uniformly mixing. Thus there was obtained the rinse type face pack as desired.

The rinse type face pack obtained as mentioned above was found to spread easily, has excellent washing effect, and impart slippery feeling after rinsing.

### Example 36

### Deodorant

| | Formulation | weight % |
|---|---|---|
| 1. | Dimethylpolysiloxane (6 mm²/s at 25° C.) | 10.0 |
| 2. | Decamethylcyclopentasiloxane | 2.0 |
| 3. | Solution of polymer-A in D5 | 4.0 |
| 4. | Branched polyglycerinether-modified silicone (*1) | 1.0 |
| 5. | Sorbitan monostearate | 1.0 |
| 6. | Penthylene glycol | 30.0 |
| 7. | Glycerin | 15.0 |
| 8. | Trichlosan | 0.1 |
| 9. | Sodium chloride | 0.1 |
| 10. | Antiseptic | q.s. |
| 11. | Purified water | 36.8 |
| | Total | 100.0 |

*1: KF-6100 from Shin-Etsu Chemical Co., Ltd.

### <Preparation of Cosmetic Product>

A: prepared from ingredients 1 to 5 by uniform mixing.
B: prepared from ingredients 6 to 11 by uniform mixing.
C: prepared by adding A to B under agitation and mixing. Thus there was obtained the deodorant as desired.

The deodorant obtained as mentioned above was found to spread easily without sticky feeling and oily feeling, and keep the deodorant effect.

### Example 37

### O/W/O Milky Lotion

| | Formulation | weight % |
|---|---|---|
| 1. | Crosslinked polyether-modified silicone (*1) | 3.0 |
| 2. | Branched polyether-modified silicone (*2) | 1.0 |
| 3. | Solution of polymer-A in D5 | 14.0 |
| 4. | Crosslinked alkyl-modified silicone compound (*3) | 5.0 |
| 5. | Sucrose stearate | 3.0 |
| 6. | 1,3-butylene glycol | 5.0 |
| 7. | Glycerin | 5.0 |
| 8. | Antiseptic | q.s. |
| 9. | Purified water | 60.0 |
| 10. | Macademian nut oil | 2.0 |
| 11. | Cetyl alcohol | 2.0 |
| 12. | Perfume | q.s. |
| | Total | 100.0 |

*1: KSG-210 from Shin-Etsu Chemical Co., Ltd.
*2: KF-6028 from Shin-Etsu Chemical Co., Ltd.
*3: KSG-43 from Shin-Etsu Chemical Co., Ltd.

### <Preparation of Cosmetic Product>

A: prepared from ingredients 1 to 4 by uniform mixing.
B: prepared from ingredients 5 to 9 by uniform mixing under heating.
C: prepared from ingredients 10 to 12 by mixing under heating.
D: prepared by slowly adding C to B with stirring for emulsification, and then cooling.
E: prepared by slowly adding D to A with stirring for emulsification. Thus there was obtained the O/W/O milky lotion as desired.

The O/W/O milky lotion obtained as mentioned above was found to spread easily without oily feeling and to keep cool feeling.

### Example 38

### O/W/O Liquid Foundation

| | Formulation | weight % |
|---|---|---|
| 1. | Crosslinked polyether-modified silicone (*1) | 4.0 |
| 2. | Branched polyether-modified silicone (*2) | 1.0 |
| 3. | Solution of polymer-A in D5 | 3.0 |
| 4. | Propylene glycol decanate | 5.0 |
| 5. | Isopropyl myristate | 3.0 |
| 6. | Foundation pigment (treated with silicone) (*3) | 10.0 |
| 7. | Hydrogenated phospholiped derived from a yolk | 1.0 |
| 8. | Glycerin | 2.0 |
| 9. | 1,3-butylene glycol | 10.0 |
| 10. | Antiseptic | q.s. |
| 11. | Purified water | 52.0 |
| 12. | Squalane | 5.0 |
| 13. | Cetyl alcohol | 5.0 |
| 14. | Perfume | q.s. |
| | Total | 100.0 |

*1: KSG-210 from Shin-Etsu Chemical Co., Ltd.
*2: KF-6028P from Shin-Etsu Chemical Co., Ltd.
*3: Treated with KF-9909 from Shin-Etsu Chemical Co., Ltd.

### <Preparation of Cosmetic Product>

A: prepared from ingredients 1 to 4 by uniform mixing.
B: prepared from ingredients 5 to 11 by uniform mixing under heating.
C: prepared by mixing ingredients 12 to 14 under heating.
D: prepared by slowly adding C to B with stirring for emulsification, and then cooling.
E: prepared by slowly adding D to A with stirring for emulsification. Thus there was obtained the O/W/O liquid foundation as desired.

The O/W/O liquid foundation obtained as mentioned above was found to spread lightly without sticky feeling and oily feeling, last long without secondary sticking.

### Example 39

### W/O Cream

| | Formulation | weight % |
|---|---|---|
| 1. | Crosslinked polyether-modified silicone (*1) | 3.0 |
| 2. | Crosslinked dimethylpolysiloxane (*2) | 20.0 |
| 3. | Crosslinked dimethylpolysiloxane (*3) | 6.0 |
| 4. | Branched polyether-modified silicone (*4) | 1.0 |
| 5. | Decamethylcyclopentasiloxane | 15.0 |
| 6. | Dimethylpolysiloxane (6 mm²/s at 25° C.) | 4.0 |
| 7. | Solution of polymer-A in D5 | 6.0 |
| 8. | Dimethyldistearylammoniumhectorite | 2.0 |
| 9. | 1,3-butylene glycol | 10.0 |
| 10. | Sodium chloride | 0.1 |
| 11. | Antiseptic | q.s. |
| 12. | Perfume | q.s. |
| 13. | Purified water | 32.9 |
| | Total | 100.0 |

*1: KSG-210 from Shin-Etsu Chemical Co., Ltd.
*2: KSG-15 from Shin-Etsu Chemical Co., Ltd.
*3: KSG-16 from Shin-Etsu Chemical Co., Ltd.
*4: KF-6028P from Shin-Etsu Chemical Co., Ltd.

### <Preparation of Cosmetic Product>

A: prepared from ingredients 1 to 8 by uniform mixing with heating at 50° C.
B: prepared from ingredients 9 to 11 and 13 by uniform mixing.
C: prepared by slowly adding B to A with stirring for emulsification. Then, ingredient 12 was added. Thus there was obtained the W/O cream as desired.

The W/O cream obtained as mentioned above was found to spread easily and keep the skin moist without sticky feeling and oily feeling.

### Example 40

### W/O Blusher

| | Formulation | weight % |
|---|---|---|
| 1. | Acrylsilicone resin solution (*1) | 10.0 |
| 2. | Stearyl-modified acrylsilicone resin (*2) | 2.0 |
| 3. | Branched polyether-modified silicone (*3) | 1.5 |
| 4. | Decamethylcyclopentasiloxane | 15.0 |
| 5. | Glyceryl triisostearate | 3.0 |
| 6. | Solution of polymer-A in D5 | 5.0 |
| 7. | Dimethyldistearylammoniumhectorite | 1.5 |
| 8. | Spherical nylon powder | 3.0 |
| 9. | Talc | 4.0 |
| 10. | Blusher pigment (treated with acryl-silicone) (*4) | 20.0 |
| 11. | Alcohol | 5.0 |
| 12. | Perfume | q.s. |
| 13. | Purified water | 30.0 |
| | Total | 100.0 |

*1: KP-545 from Shin-Etsu Chemical Co., Ltd.
*2: KP-561P from Shin-Etsu Chemical Co., Ltd.
*3: KF-6028P from Shin-Etsu Chemical Co., Ltd.
*4: treated with KP-574 from Shin-Etsu Chemical Co., Ltd.

### <Preparation of Cosmetic Product>

A: prepared from ingredients 1 to 7 by mixing with heating.
B: prepared from ingredients 8 to 10 and 12 by uniform mixing, followed by mixing with A.
C: prepared from ingredients 11 and 13 by mixing.
D: prepared by slowly adding B to C with stirring for emulsification. Thus there was obtained the W/O blusher as desired.

The W/O blusher obtained as mentioned above was found to spread easily, last long, and adhere well without sticky feeling and oily feeling.

### Example 41

### W/O Liquid Foundation

| | Formulation | weight % |
|---|---|---|
| 1. | Crosslinked polyether-modified silicone (*1) | 3.0 |
| 2. | Crosslinked dimethylpolysiloxane (*2) | 5.0 |
| 3. | Branched polyether-modified silicone (*3) | 2.0 |
| 4. | Decamethylcyclopentasiloxane | 18.0 |
| 5. | Cetyl isooctanoate | 5.0 |
| 6. | Solution of polymer-A in D5 | 12.0 |
| 7. | Dimethyldistearylammoniumhectorite | 1.2 |
| 8. | Foundation pigment (silicone-treated) (*4) | 14.0 |
| 9. | Acrylsilicone resin solution (*5) | 10.0 |
| 10. | 1,3-butylene glycol | 5.0 |
| 11. | Xanthan gum | 0.1 |
| 12. | Sodium citrate | 0.2 |
| 13. | Sodium chloride | 0.5 |
| 14. | Antiseptic | q.s. |
| 15. | Perfume | q.s. |
| 16. | Purified water | 24.0 |
| | Total | 100.0 |

*1: KSG-210 from Shin-Etsu Chemical Co., Ltd.
*2: KSG-15 from Shin-Etsu Chemical Co., Ltd.
*3: KF-6028P from Shin-Etsu Chemical Co., Ltd.
*4: treated with KF-9909 from Shin-Etsu Chemical Co., Ltd.
*5: KP-575 from Shin-Etsu Chemical Co., Ltd.

### <Preparation of Cosmetic Product>

A: prepared from a portion of ingredient 4 and ingredient 9 by mixing. Then ingredient 8 was uniformly dispersed into it.
B: prepared from ingredients 1 to 3, the remainder of ingredient 4, and ingredients 5 to 7 by uniform mixing.
C: prepared from ingredients 10 to 14 and 16 by uniform mixing.
D: prepared by slowly adding B to C with stirring for emulsification. A and ingredient 15 were added. Thus there was obtained the W/O liquid foundation as desired.

The W/O liquid foundation obtained as mentioned above was found to spread easily and last long without sticky feeling and oily feeling and secondary sticking, as well as to have excellent stability.

### Example 42

### W/O Cream

| | Formulation | weight % |
|---|---|---|
| 1. | Crosslinked alkyl•polyether-modified silicone (*1) | 3.0 |
| 2. | Crosslinked alkyl-modified dimethylpolysiloxane (*2) | 4.0 |
| 3. | Alkyl-modified branched polyether-modified silicone (*3) | 1.0 |
| 4. | Meadowfoam oil | 3.5 |
| 5. | Jojoba oil | 2.5 |
| 6. | Macademianut oil | 5.0 |
| 7. | Solution of polymer-A in D5 | 7.5 |
| 8. | Hybrid silicone complex powder (*4) | 3.0 |
| 9. | 1,3 -butylene glycol | 8.0 |
| 10. | Glycine | 3.0 |
| 11. | Sodium citrate | 0.2 |
| 12. | Sodium chloride | 0.5 |
| 13. | Antiseptic | q. s . |
| 14. | Perfume | q. s . |
| 15. | Purified water | 58.8 |
| | Total | 100.0 |

*1: KSG-340 from Shin-Etsu Chemical Co., Ltd.
*2: KSG-44 from Shin-Etsu Chemical Co., Ltd.
*3: KF-6038 from Shin-Etsu Chemical Co., Ltd.
*4: KSP-100 from Shin-Etsu Chemical Co., Ltd.

### <Preparation of Cosmetic Product>

A: prepared from ingredients 1 to 8 by uniform mixing.
B: prepared from ingredients 9 to 13 and 15 by uniform mixing.
C: prepared by slowly adding B to A with stirring for emulsification. Then, ingredient 14 was added. Thus there was obtained the W/O cream as desired.

The W/O cream obtained as mentioned above was found to keep the skin moist without sticky feeling and oily feeling.

### Example 43

### Lipstick

| | Formulation | weight % |
|---|---|---|
| 1. | Candelilla wax | 4.0 |
| 2. | Polyethylene | 2.0 |
| 3. | Microcrystalline wax | 3.0 |
| 4. | Seresin | 7.0 |
| 5. | Stearyl-modified acrylsilicone resin (*1) | 15.0 |
| 6. | Diphenyldimethicone (*2) | 20.0 |
| 7. | Solution of polymer-A in D5 | 10.0 |
| 8. | Alkyl-modified branched polyglycerin-modified silicone(*3) | 3.0 |
| 9. | Macademianut oil | 20.0 |
| 10. | Hydrogenated polyisobutene | 10.0 |
| 11. | Isotridecyl isononanonate | 6.0 |
| 12. | Perfume | q.s. |
| 13. | Lipstick pigment | q.s. |
| 14. | Mica | q.s. |
| | Total | 100.0 |

*1: KP-561P from Shin-Etsu Chemical Co., Ltd.
*2: KF-54 from Shin-Etsu Chemical Co., Ltd.
*3: KF-6105 from Shin-Etsu Chemical Co., Ltd.

### <Preparation of Cosmetic Product>

A: prepared from ingredients 1 to 12 by uniform mixing with heating.
B: prepared by adding ingredients 13 and 14 to A, followed by uniform mixing with heating. The resulting mixture was filled into air-tight containers. Thus, there was obtained the lipstick as desired.

The lipstick obtained as mentioned above was found to last long without blotting, sticky feeling, and oily feeling, as well as to impart good luster to hair.

### Example 44

### Cuticle Coat

| | Formulation | weight % |
|---|---|---|
| 1. | Polyether-modified silicone (*1) | 3.0 |
| 2. | Polyether-modified silicone (*2) | 2.0 |
| 3. | PEG-40 hydrogenated hardened castor oil | 1.0 |
| 4. | Solution of polymer-A in D5 | 3.0 |
| 5. | Silicone gum solution (*3) | 40.0 |
| 6. | Decamethylcyclopentasiloxane | 40.0 |
| 7. | Alcohol | 5.0 |
| 8. | Antiseptic | q.s. |
| 9. | Perfume | q.s. |
| 10. | Purified water | 6.0 |
| | Total | 100.0 |

*1: KF-6011 from Shin-Etsu Chemical Co., Ltd.
*2: KF-6013 from Shin-Etsu Chemical Co., Ltd.
*3: KF-9028 from Shin-Etsu Chemical Co., Ltd.

### <Preparation of Cosmetic Product>

A: prepared from ingredients 1 to 3 and 7 to 10 by uniform mixing.
B: prepared from ingredients 4 to 6 by uniform mixing.
C: prepared by adding B to A with stirring for emulsification. Thus there was obtained the cuticle coat as desired.

The cuticle coat obtained as mentioned above was found to spread easily and make the hair lustrous and smooth while preventing the hair from becoming dry and loose.

### Example 45

### Hair Treatment

| | Formulation | weight % |
|---|---|---|
| 1. | Silicone gum solution (*1) | 5.0 |
| 2. | Diphenyldimethicone (*2) | 4.0 |
| 3. | Solution of polymer-A in D5 | 1.0 |
| 4. | Cetyl octanoate | 1.0 |
| 5. | Cetyl alcohol | 0.5 |
| 6. | Polyether-modified silicone (*3) | 1.0 |
| 7. | PEG-60 hydrogenated hardened castor oil | 1.0 |
| 8. | Glyceryl monostearate | 0.5 |
| 9. | Carboxyvinylpolymer (1 wt % aqueous solution) | 25.0 |
| 10. | Xanthan gum (1 wt % aqueous solution) | 7.0 |
| 11. | 1,3-butylene glycol | 5.0 |
| 12. | Alcohol | 7.0 |
| 13. | Antiseptic | q.s. |
| 14. | Perfume | q.s. |
| 15. | Purified water | 42.0 |
| | Total | 100.0 |

*1: MK-15H from Shin-Etsu Chemical Co., Ltd.
*2: KF-54 from Shin-Etsu Chemical Co., Ltd.
*3: KF-6013 from Shin-Etsu Chemical Co., Ltd.

### <Preparation of Cosmetic Product>

A: prepared from ingredients 1 to 8 by dissolution with heating.
B: prepared from ingredients 11 to 15 by dissolution with heating.
C: prepared by adding B to A with stirring for emulsification. Ingredients 9 and 10 were added. Thus there was obtained the hair treatment as desired.

The hair treatment obtained as mentioned above was found to spread easily and make the hair lustrous and smooth.

### Example 46

### Nail Enamel

| | Formulation | weight % |
|---|---|---|
| 1. | Solution of polymer-A in M3T | 45.0 |
| 2. | Methyltrimethicone (*1) | 5.0 |
| 3. | Nitrocellulose | 3.0 |
| 4. | Camphor | 0.5 |
| 5. | Acetyltributyl citrate | 1.0 |
| 6. | Dimethyldistearylammoniumhectorite | 0.5 |
| 7. | Butyl acetate | 30.0 |
| 8. | Ethyl acetate | 10.0 |
| 9. | Isopropyl alcohol | 5.0 |
| 10. | Colored pigment | q.s. |
| | Total | 100.0 |

*1: TMF-1.5 from Shin-Etsu Chemical Co., Ltd.

### <Preparation of Cosmetic Product>

A: prepared from ingredients 7 to 9 by mixing. Ingredients 4 to 6 were added, followed by uniform mixing.
B: prepared by adding ingredients 1 to 3 to A, followed by mixing.
C: prepared by adding ingredient 10 to B. Thus there was obtained the nail enamel as desired.

The nail enamel obtained as mentioned above was found to spread easily, and impart luster and good adhesion to the nail.

### Example 47

### Nail Enamel Overcoat

| | Formulation | weight % |
|---|---|---|
| 1. | Solution of polymer-A in isododecane | 6.0 |
| 2. | Nitrocellulose | 17.0 |
| 3. | Alkyd resin | 4.0 |
| 4. | Acetyltriethyl citrate | 5.0 |
| 5. | Butyl acetate | 29.0 |
| 6. | Ethyl acetate | 25.0 |
| 7. | Isopropyl alcohol | 3.0 |
| 8. | n-butyl alcohol | 1.0 |
| 9. | Toluene | 10.0 |
| | Total | 100.0 |

### <Preparation of Cosmetic Product>

A: prepared from ingredients 5 to 9 by mixing. Ingredient 4 was added, followed by uniform mixing.
B: prepared by adding ingredients 1 to 3 to A, followed by mixing. Thus there was obtained the nail enamel overcoat as desired.

The nail enamel overcoat obtained as mentioned above was found to spread easily, make the enamel more lustrous, and impart good adhesion to nails.

1. A cosmetic product comprising a polymer comprising recurring units of formula (1): 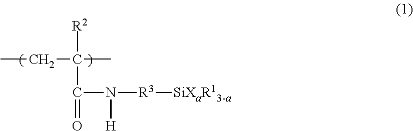
wherein R¹ is independently an alkyl group or a fluorinated alkyl group having 1 to 12 carbon atoms, R² is methyl group or hydrogen atom, R³ is a divalent saturated hydrocarbon group having 1 to 6 carbon atoms, letter a is an integer of 1 to 3, and X is a monovalent group represented by formula (i): 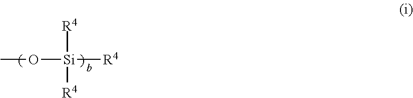
wherein R⁴ is independently an alkyl group or a fluorinated alkyl group having 1 to 12 carbon atoms and letter b is an integer of 1 to 5, and
recurring units of formula (2) 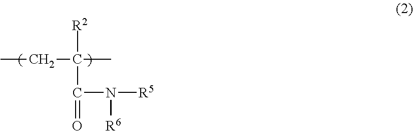
wherein R² is methyl group or hydrogen atom, and R⁵ and R⁶ are independently hydrogen atom, an alkyl group, a fluorinated alkyl group, a hydroxyalkyl group, or a monovalent organic group containing a (poly)glyceryl group, or R⁵ and R⁶ may bond together to form a ring with the carbon nitrogen to which they are attached, and when the ring is formed, —R⁵—R⁶— is an alkylene group which may contain an ether bond (—O—), and/or
recurring units of formula (3) 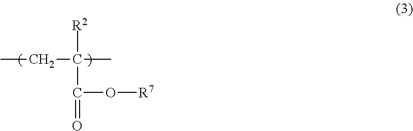
wherein R² is methyl group or hydrogen atom, and R⁷ is hydrogen atom, an alkyl group, a fluorinated alkyl group, a hydroxyalkyl group, or a monovalent organic group containing a (poly)oxyalkylene group, (poly)glyceryl group or siloxane bond.

2. The cosmetic product of claim 1 wherein the polymer has a number average molecular weight of 300 to 1,000,000 by GPC using polystyrene standards.

3. The cosmetic product of claim 1 wherein the polymer is present in an amount of 0.01 to 10% by weight of the total weight of the cosmetic product.

4. The cosmetic product of claim 3 wherein the polymer is present in an amount of 0.1 to 7.5% by weight of the total weight of the cosmetic product.

5. The cosmetic product of claim 1 which further comprises a linear, branched or cyclic silicone oil in a weight ratio of the silicone oil and the polymer of 1:2 to 100:1.

6. The cosmetic product of claim 5 wherein the silicone oil is volatile at 25° C.

7. The cosmetic product of claim 6 wherein the silicone oil is at least one selected from the group consisting of hexamethyldisiloxane, octamethyltrisiloxane, decamethyltetrasiloxane, dodecamethylpentasiloxane, octamethylcyclotetrasiloxane, decamethylcyclopentasiloxane, tristrimethylsiloxymethylsilane, and tetrakistrimethylsiloxysilane.

8. The cosmetic product of claim 1, which further comprises one or more oils selected from the group consisting of silicone oils, silicone gums, hydrocarbon oils, higher fatty acids, higher alcohol oils, ester oils, glyceride oils, animal and vegetable oils, semi-synthetic oils, and fluorine-derived oils.

9. The cosmetic product of claim 1, which further comprises a sugar alcohol, sterol, one or more water-soluble monohydric alcohols or water-soluble polyhydric alcohol alcohols.

10. The cosmetic product of claim 1, which further comprises one or more water-soluble polymeric compounds or water-swelling polymeric compounds.

11. The cosmetic product of claim 1, which further comprises from 1 to 99 weight-% water, based on the total weight of the cosmetic product.

12. The cosmetic product of claim 1, which further comprises one or more inorganic powders, powder selected from the group consisting of organic powders, metal soaps, powders of metal salts of surface active agents, colored pigments, dyes, pearlescent pigments, metal powders powder pigments, and tar dyes, or natural colors.

13. The cosmetic product of claim 1, which further comprises one or more cosmetically acceptable anionic, cationic, nonionic, or amphoteric surface active agents.

14. The cosmetic product of claim 1, formulated for application to hair.

15. The cosmetic product of claim 1, formulated as a lipstick.

16. The cosmetic product of claim 1, formulated as a nail enamel.
